(19) United States
(12) Patent Application Publication
(10) Pub. No.: US 2013/0266565 A1
(43) Pub. Date: Oct. 10, 2013

(54) ANTI-MHC ANTIBODY ANTI VIRAL CYTOKINE FUSION PROTEIN

(71) Applicant: F. Hoffmann-La Roche AG, Basel (CH)

(72) Inventors: Stefan Jenewein, Neustadt/Weinstrasse (DE); Erhard Kopetzki, Penzberg (DE); Stefan Ries, Penzberg (DE); Georg Tiefenthaler, Sindelsdorf (DE)

(21) Appl. No.: 13/758,788

(22) Filed: Feb. 4, 2013

Related U.S. Application Data
(63) Continuation of application No. PCT/EP2011/063362, filed on Aug. 3, 2011.

(30) Foreign Application Priority Data
Aug. 5, 2010 (EP) 10172054.8
Nov. 17, 2010 (EP) 10191498.4

(51) Int. Cl. C07K 16/08

(52) U.S. Cl. CPC C07K 16/082
USPC 424/134.1; 530/387.3; 536/23.4; 435/328; 435/69.6

(57) ABSTRACT

The invention provides a fusion protein comprising an antibody that binds to a human major histocompatibility complex presenting a peptidic fragment of a hepatitis-B-virus protein and an anti-viral cytokine and methods of using the same.

The application is a continuation of International Application No. PCT/EP2011/063362, filed 3 Aug. 2011, which claims priority to EP Application Nos 10172054.8, filed 5 Aug. 2010 and 10191498.4, filed 17 Nov. 2010, the contents of each of which are hereby incorporated by reference in their entirety.

### SEQUENCE LISTING

The instant application contains a Sequence Listing submitted via EFS-Web and hereby incorporated by reference in its entirely. Said ASCII copy, created on Feb. 4, 2013, is named P4660Cl_SeqList.txt and is 45,820 bytes in size.

### FIELD OF THE INVENTION

The present invention relates to fusion proteins comprising an antibody that binds to a human major histocompatibility complex presenting a peptidic fragment of a hepatitis-B-virus protein and an anti-viral cytokine and methods of using the same. The fusion protein can be used for the treatment of viral infections, such as hepatitis-B-virus infections.

### BACKGROUND

HBV is susceptible to the antiviral effect of type I and type II interferons but the effectiveness of these cytokines during chronic HBV infection is reduced, as chronic HBV is associated with suppressed anti-viral innate and adaptive immune responses. To circumvent these immune defects and increase the efficacy of current interferon therapy against chronic HBV infection we created a novel tool that combines the exquisite specificity of HBV-specific CD8 T cells with the antiviral effect of cytokines in a format resistant to the hepatic suppression.

Interferon, in particular interferon 2α, is a pharmaceutically active protein which has anti-viral and anti-proliferative activity. For example interferon is used to treat hairy cell leukemia and Kaposi's sarcoma, and is active against hepatitis. In order to improve stability and solubility, and reduce immunogenicity, pharmaceutically active proteins such as interferon may be conjugated to the polymer polyethylene glycol (PEG) (see EP 0 809 996).

### SUMMARY

It has been found that the fusion protein as reported herein can deliver interferon-alpha to HBV-infected target cells with greater potency than naked or PEGylated interferon. The fusion protein as reported herein is a novel targeted therapeutic delivery platform to provide a treatment for HBV-infected patients with potentially reduced pleiotropic effects of interferon.

The invention provides a fusion protein comprising an antibody that binds to a human major histocompatibility complex presenting a peptidic fragment of a hepatitis-B-virus protein and a cytokine. In one embodiment the cytokine is an anti-viral cytokine.

In one embodiment the hepatitis-B-virus protein is the hepatitis-B-virus envelope (env) protein or the hepatitis-B-virus core protein.

In one embodiment the hepatitis-B-virus protein is the hepatitis-B-virus envelope (surface) protein and the peptidic fragments corresponds to amino acid residues 172 to 180 thereof, or the hepatitis-B-virus protein is the hepatitis-B-virus envelope (surface) protein and the peptidic fragments corresponds to amino acid residues 183 to 191 thereof, or the hepatitis-B-virus protein is the hepatitis-B-virus core protein and the peptidic fragments corresponds to amino acid residues 18 to 27 thereof. In one embodiment the peptidic fragment has the amino acid sequence of amino acid residues 172 to 180 of SEQ ID NO: 01, or has the amino acid sequence of amino acid residues 182 to 190 of SEQ ID NO: 01, or has the amino acid sequence of amino acid residues 18 to 27 of SEQ ID NO: 02. In one embodiment the peptidic fragment has the amino acid sequence of SEQ ID NO: 30, or the peptidic fragment has the amino acid sequence of SEQ ID NO: 31.

In one embodiment the antibody specifically binds to hepatocytes of subjects infected with the hepatitis-B-virus.

In one embodiment the anti-viral cytokine is an interferon. In one embodiment the anti-viral cytokine is a variant of a naturally occurring anti-viral cytokine. In one embodiment the variant is a truncated version of a naturally occurring cytokine or an anti-viral cytokine that has a consensus amino acid sequence. In a further embodiment the anti-viral cytokine is selected from type I interferon, or type II interferon, or type III interferon. In one embodiment the interferon is human interferon α-2a. In also an embodiment the anti-viral cytokine is a truncated variant of human interferon α-2a. In one embodiment the interferon has the amino acid sequence of SEQ ID NO: 03, or is a fragment thereof with comparable biological activity of the polypeptide of SEQ ID NO: 03.

In one embodiment the fusion protein has the same specificity as CD 8 bearing T-cells.

In one embodiment the antibody is not inhibited by serum hepatitis-B-virus antigens.

In one embodiment the antibody is a monoclonal antibody.

In one embodiment the antibody is a human, humanized, or chimeric antibody.

In one embodiment the antibody is an antibody fragment that binds a human major histocompatibility complex presenting a peptidic fragment of a hepatitis-B-virus protein.

In one embodiment the cytokine is fused to the N-terminus or the C-terminus of the antibody's light or heavy chain. In one embodiment the cytokine is fused to the C-terminus of the antibody's heavy chain.

In one embodiment the antibody that binds to a human major histocompatibility complex presenting a peptidic fragment of a hepatitis-B-virus protein and the anti-viral cytokine are fused via a linker peptide. In one embodiment the linker peptide is selected from SEQ ID NO: 22 to SEQ ID NO: 27. In one embodiment the linker peptide has the amino acid sequence of SEQ ID NO: 22.

In one embodiment the antibody comprises (a) HVR-H3 comprising the amino acid sequence of SEQ ID NO: 06, (b) HVR-L3 comprising the amino acid sequence of SEQ ID NO: 10, (c) HVR-H2 comprising the amino acid sequence of SEQ ID NO: 05, or a humanized variant thereof.

In one embodiment the antibody comprises (a) HVR-H1 comprising the amino acid sequence of SEQ ID NO: 04, (b) HVR-H2 comprising the amino acid sequence of SEQ ID NO: 05, (c) HVR-H3 comprising the amino acid sequence of SEQ ID NO: 06, or a humanized variant thereof.

In one embodiment the antibody comprises (a) HVR-L1 comprising the amino acid sequence of SEQ ID NO: 08; (b) HVR-L2 comprising the amino acid sequence of SEQ ID NO: 09; (c) HVR-L3 comprising the amino acid sequence of SEQ ID NO: 10; or a humanized variant thereof.

In one embodiment the antibody comprises (a) a VH sequence having at least 95% sequence identity to the amino acid sequence of SEQ ID NO: 07, or to a humanized variant thereof; or (b) a VL sequence having at least 95% sequence identity to the amino acid sequence of SEQ ID NO: 11, or to a humanized variant thereof; or (c) a VH sequence as in (a) and a VL sequence as in (b), or a humanized variant thereof.

In one embodiment the antibody comprises a VH sequence of SEQ ID NO: 07, or a humanized variant thereof.

In one embodiment the antibody comprises a VL sequence of SEQ ID NO: 11, or a humanized variant thereof.

In one embodiment the antibody heavy chain has the amino acid sequence of SEQ ID NO: 12, or is a humanized variant thereof.

In one embodiment the antibody heavy chain has the amino acid sequence of SEQ ID NO: 13, or is a humanized variant thereof.

In one embodiment the antibody light chain has the amino acid sequence of SEQ ID NO: 14, or is a humanized variant thereof.

In one embodiment the antibody light chain has the amino acid sequence of SEQ ID NO: 15, or is a humanized variant thereof.

In one embodiment the antibody comprises (a) HVR-H3 comprising the amino acid sequence of SEQ ID NO: 34, (b) HVR-L3 comprising the amino acid sequence of SEQ ID NO: 38, (c) HVR-H2 comprising the amino acid sequence of SEQ ID NO: 33, or a humanized variant thereof.

In one embodiment the antibody comprises (a) HVR-H1 comprising the amino acid sequence of SEQ ID NO: 32, (b) HVR-H2 comprising the amino acid sequence of SEQ ID NO: 33, (c) HVR-H3 comprising the amino acid sequence of SEQ ID NO: 34, or a humanized variant thereof.

In one embodiment the antibody comprises (a) HVR-L1 comprising the amino acid sequence of SEQ ID NO: 36; (b) HVR-L2 comprising the amino acid sequence of SEQ ID NO: 37; (c) HVR-L3 comprising the amino acid sequence of SEQ ID NO: 38; or a humanized variant thereof.

In one embodiment the antibody comprises (a) a VH sequence having at least 95% sequence identity to the amino acid sequence of SEQ ID NO: 35, or to a humanized variant thereof; or (b) a VL sequence having at least 95% sequence identity to the amino acid sequence of SEQ ID NO: 39, or to a humanized variant thereof; or (c) a VH sequence as in (a) and a VL sequence as in (b), or a humanized variant thereof.

In one embodiment the antibody comprises a VH sequence of SEQ ID NO: 35, or a humanized variant thereof.

In one embodiment the antibody comprises a VL sequence of SEQ ID NO: 39, or a humanized variant thereof.

In one embodiment the antibody is a full length human IgG1 antibody.

The invention further provides an isolated nucleic acid encoding the fusion protein as reported herein. Also provided are isolated nucleic acids encoding an antibody heavy chain as reported herein. Further provided is an isolated nucleic acid encoding the antibody light chain as reported herein.

The invention also provides a host cell comprising one or more of the nucleic acids as reported herein.

Also provided is a method of producing a fusion protein as reported herein comprising culturing a host cell as reported herein so that the fusion protein is produced. In one embodiment the method comprises the following steps: (a) providing a cell as reported herein, (b) cultivating the provided cell, (c) recovering the fusion protein from the cell or the cultivation medium and thereby producing the fusion protein.

The invention provides a pharmaceutical formulation comprising the fusion protein as reported herein and a pharmaceutically acceptable carrier.

The invention further provides the fusion protein as reported herein for use as a medicament.

The invention also provides the fusion protein as reported herein for use in treating hepatitis-B-virus infection.

The invention still provides the fusion protein as reported herein for use in delivering an anti-viral cytokine to hepatitis-B-virus infected hepatocytes.

The invention also provides the use of the fusion protein as reported herein in the manufacture of a medicament. In one embodiment the medicament is for the treatment of hepatitis-B-virus infection. In a further embodiment the hepatitis-B-virus infection is a chronic infection. In also an embodiment the medicament is for delivering an anti-viral cytokine to hepatitis-B-virus infected hepatocytes.

The invention provides a method of treating an individual having a hepatitis-B-virus infection comprising administering to the individual an effective amount of the fusion protein as reported herein.

The invention also provides a method of delivering an anti-viral cytokine to hepatitis-B-virus infected hepatocytes in an individual comprising administering to the individual an effective amount of the fusion protein as reported herein to deliver an anti-viral cytokine to hepatitis-B-virus infected hepatocytes.

In alternative embodiments, the invention provides the following:
1. A fusion protein comprising an antibody that specifically binds to a human major histocompatibility complex presenting a peptidic fragment of a hepatitis-B-virus protein and an anti-viral cytokine.
2. The fusion protein according to claim 1, wherein the peptidic fragment of an hepatitis-B-virus protein has the amino acid sequence of amino acid residues 182 to 190 of SEQ ID NO: 01, or has the amino acid sequence of amino acid residues 18 to 27 of SEQ ID NO: 02.
3. The fusion protein according to any one of the preceding claims, wherein the antibody specifically binds to hepatocytes infected with hepatitis-B-virus.
4. The fusion protein according to any one of the preceding claims, wherein the anti-viral cytokine is selected from type I and/or type II interferons.
5. The fusion protein according to any one of the preceding claims, wherein the fusion protein has the same specificity as CD 8 bearing T-cells.
6. The fusion protein according to any one of the preceding claims, wherein the antibody does not specifically bind to serum hepatitis-B-virus antigens.
7. The fusion protein according to any one of the preceding claims, wherein the antibody is a monoclonal antibody.
8. The fusion protein according to any one of the preceding claims, wherein the antibody is a human, humanized, or chimeric antibody.
9. The fusion protein according to any one of the preceding claims, wherein the antibody is an antibody fragment that binds a human major histocompatibility complex presenting a peptidic fragment of a hepatitis-B-virus protein.
10. The fusion protein according to any one of the preceding claims, wherein the antibody comprises (a) CDR-H3 comprising the amino acid sequence of SEQ ID NO: 06, (b) CDR-L3 comprising the amino acid sequence of SEQ ID NO: 10, and (c) CDR-H2 comprising the amino acid sequence of SEQ ID NO: 05, or wherein the antibody comprises (a) CDR-H3 comprising the amino acid sequence of SEQ ID NO: 34, (b) CDR-L3 comprising the amino acid sequence of SEQ ID NO: 38, and (c) CDR-H2 comprising the amino acid sequence of SEQ ID NO: 33.
11. The fusion protein according to any one of the preceding claims, wherein the antibody comprises (a) CDR-H1 comprising the amino acid sequence of SEQ ID NO: 04, (b) CDR-H2 comprising the amino acid sequence of SEQ ID NO: 05, and (c) CDR-H3 comprising the amino acid sequence of SEQ ID NO: 06, or wherein the antibody comprises (a) CDR-H1 comprising the amino acid sequence of SEQ ID NO: 32, (b) CDR-H2 comprising the amino acid sequence of SEQ ID NO: 33, and (c) CDR-H3 comprising the amino acid sequence of SEQ ID NO: 34.
12. The fusion protein according to any one of the preceding claims, wherein the antibody comprises (a) CDR-L1 comprising the amino acid sequence of SEQ ID NO: 08; (b) CDR-L2 comprising the amino acid sequence of SEQ ID NO: 09; and (c) CDR-L3 comprising the amino acid sequence of SEQ ID NO: 10, or wherein the antibody comprises (a) CDR-L1 comprising the amino acid sequence of SEQ ID NO: 36; (b) CDR-L2 comprising the amino acid sequence of SEQ ID NO: 37; and (c) CDR-L3 comprising the amino acid sequence of SEQ ID NO: 38.
13. The fusion protein according to any one of the preceding claims, wherein the antibody comprises
   (i) a VH sequence having at least 95% sequence identity to the amino acid sequence of SEQ ID NO: 07 or to a humanized variant thereof;
      a VL sequence having at least 95% sequence identity to the amino acid sequence of SEQ ID NO: 11 or to a humanized variant thereof; or
      a VH sequence having at least 95% sequence identity to the amino acid sequence of SEQ ID NO: 07 and a VL sequence having at least 95% sequence identity to the amino acid sequence of SEQ ID NO: 11, or to a humanized variant thereof,
   or
   (ii) a VH sequence having at least 95% sequence identity to the amino acid sequence of SEQ ID NO: 35 or to a humanized variant thereof;
      a VL sequence having at least 95% sequence identity to the amino acid sequence of SEQ ID NO: 39 or to a humanized variant thereof; or
      a VH sequence having at least 95% sequence identity to the amino acid sequence of SEQ ID NO: 35 and a VL sequence having at least 95% sequence identity to the amino acid sequence of SEQ ID NO: 39, or to a humanized variant thereof.
14. The fusion protein according to any one of the preceding claims, wherein the antibody comprises a VH sequence of SEQ ID NO: 07, or of SEQ ID NO: 35, or a humanized variant thereof.
15. The fusion protein according to any one of the preceding claims, wherein the antibody comprises a VL sequence of SEQ ID NO: 11, or of SEQ ID NO: 39, or a humanized variant thereof.
16. The fusion protein according to any one of the preceding claims, wherein one or two antibody heavy chain(s) has/have the amino acid sequence of SEQ ID NO: 13.
17. The fusion protein according to any one of the preceding claims, wherein one or two antibody light chain(s) has/have the amino acid sequence of SEQ ID NO: 14.
18. The fusion protein according to any one of the preceding claims, wherein one or two antibody light chain(s) has/have the amino acid sequence of SEQ ID NO: 15.
19. The fusion protein according to any one of the preceding claims, wherein the antibody is a full length human IgG1 antibody, or comprises a truncated human gamma-1 heavy chain constant region.
20. Isolated nucleic acid encoding the fusion protein of claim 1.
21. Isolated nucleic acid encoding an antibody chain of claim 16 or 18.
22. Isolated nucleic acid encoding the antibody light chain of claim 17.
23. A host cell comprising the nucleic acid of any one of claims 20, or 21 and 22.
24. A method of producing a fusion protein comprising culturing a host cell of claim 23 so that the fusion protein is produced.
25. The method according to claim 24 comprising the following steps:
   (a) providing a cell according to claim 23,
   (b) cultivating the provided cell,
   (c) recovering the fusion protein from the cell or the cultivation medium and thereby producing the fusion protein.
26. A pharmaceutical formulation comprising the fusion protein of any one of claims 1 to 19 and a pharmaceutically acceptable carrier.
27. The fusion protein of any one of claims 1 to 19 for use as a medicament.
28. The fusion protein of any one of claims 1 to 19 for use in treating hepatitis-B-virus infection.
29. The fusion protein of any one of claims 1 to 19 for use in delivering an anti-viral cytokine to hepatitis-B-virus infected hepatocytes.
30. Use of the fusion protein of any one of claims 1 to 19 in the manufacture of a medicament.
31. The use of claim 30, wherein the medicament is for the treatment of hepatitis-B-virus infection.
32. The use of claim 31, wherein the hepatitis-B-virus infection is a chronic hepatitis-B-virus infection.
33. The use of claim 30, wherein the medicament is for delivering an anti-viral cytokine to hepatitis-B-virus infected hepatocytes.
34. A method of treating an individual having a hepatitis-B-virus infection comprising administering to the individual an effective amount of the fusion protein of any one of claims 1 to 19.
35. A method of delivering an anti-viral cytokine to hepatitis-B-virus infected hepatocytes in an individual comprising administering to the individual an effective amount of the fusion protein of any one of claims 1 to 19 to deliver an anti-viral cytokine to hepatitis-B-virus infected hepatocytes.

### BRIEF DESCRIPTION OF THE FIGURES

FIG. 1 shows the SPR binding curves determined (a) for interferon α-2a and (b) for an antibody (human Fc-region)-interferon α-2a fusion protein (example 3).

FIG. 2 shows the plasmid map of the heavy chain expression plasmid 9924 (Example 1).

FIG. 3 shows the plasmid map of the light chain expression plasmid 9922 (Example 1).

FIG. 4 shows the normalized RLU obtained with different interferon α-2a variants.

FIG. 5 shows the binding specificity of different antibodies to HBV-infected cells; tested antibodies in both panels: i) antibody that binds to a human major histocompatibility complex presenting the peptidic fragment of SEQ ID NO: 30 of a hepatitis-B-virus protein, ii) antibody that binds to a human major histocompatibility complex presenting a peptidic fragment of SEQ ID NO: 31 of a hepatitis-B-virus protein, iii) two different anti-MAGE antibodies, iv) two different anti-HBV antibodies, v) an anti-hCMV antibody, vi) two different anti-EBV antibodies, and vii) two different anti-influenza virus antibodies; in Figure (A) only the antibody that binds to a human major histocompatibility complex presenting the peptidic fragment of SEQ ID NO: 31 of a hepatitis-B-virus protein shows binding; in Figure (B) only the antibody that binds to a human major histocompatibility complex presenting the peptidic fragment of SEQ ID NO: 30 of a hepatitis-B-virus protein shows binding.

FIG. 6 shows the recognition of peptide-MHC complexes on the surface of infected hepatocytes (HepG2 cells) by (A) i) antibody that binds to a human major histocompatibility complex presenting the peptidic fragment of SEQ ID NO: 31 of a hepatitis-B-virus protein, ii) antibody that binds to a human major histocompatibility complex presenting a peptidic fragment of SEQ ID NO: 30 of a hepatitis-B-virus protein.

FIG. 7 shows the recognition of peptide-MHC complexes on HBV infected hepatocytes of liver biopsies.

FIG. 8 shows that the fusion protein as reported herein retains its binding for HBV expressing target cells; 1: control antibody; 2: control peptide; 3: fusion protein comprising interferon-alpha and an antibody that binds to a human major histocompatibility complex presenting a peptidic fragment of a hepatitis-B-virus protein; 4: antibody that binds to a human major histocompatibility complex presenting a peptidic fragment of a hepatitis-B-virus protein.

FIG. 9 shows that the pre-blocking with the peptide of SEQ ID NO: 30 abrogates the enhanced interferon-alpha activity as shown in FIG. 8.

### DETAILED DESCRIPTION OF EMBODIMENTS OF THE INVENTION

### I. Definitions

An “acceptor human framework” denotes a human antibody framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “affinity” denotes the sum total of non-covalent interactions between a single binding site of a molecule (e.g., an antibody) and its binding partner (e.g., an antigen). The affinity of a molecule X for its partner Y can generally be represented by the dissociation constant (KD). Affinity can be determined by common methods known in the art, including those described herein.

An “affinity matured” antibody refers to an antibody with one or more alterations in one or more hypervariable regions (HVRs) or complementarity determining regions (CDRs), compared to a parent antibody which does not possess such alterations, such alterations resulting in an improvement in the affinity of the antibody for antigen, i.e. a reduction of the dissociation constant between an antibody binding site and its binding partner (antigen).

The term “amino acid” denotes the group of carboxy α-amino acids, which directly or in form of a precursor can be encoded by a nucleic acid. The individual amino acids are encoded by nucleic acids consisting of three nucleotides, so called codons or base-triplets. Each amino acid is encoded by at least one codon. This is known as “degeneration of the genetic code”. The term “amino acid” as used within this application denotes the naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “antibody that binds to a human major histocompatibility complex presenting a peptidic fragment of an hepatitis-B-virus protein” refers to an antibody that is capable of binding a human major histocompatibility complex presenting a peptidic fragment of an hepatitis-B-virus protein with sufficient affinity such that the antibody is useful as a diagnostic and/or therapeutic agent in targeting cells displaying a human major histocompatibility complex presenting a peptidic fragment of an hepatitis-B-virus protein. In certain embodiments, an antibody that binds to a human major histocompatibility complex presenting a peptidic fragment of an hepatitis-B-virus protein has a dissociation constant (Kd) of ≦10 nM, ≦1 nM, ≦0.1 nM, ≦0.01 nM, or ≦0.001 nM (e.g. 10⁻⁸M or less, e.g. from 10⁻⁸ M to 10⁻¹³M, e.g., from 10⁻⁹M to 10⁻¹³ M).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Naturally occurring antibodies are molecules with varying structures. For example, native IgG antibodies are hetero tetrameric glycoproteins of about 150,000 Daltons, composed of two identical light chains and two identical heavy chains that are disulfide-bonded. From N- to C-terminus, each heavy chain has a variable domain (VH), also called a variable heavy domain or a heavy chain variable domain, followed by three or four constant domains (CH1, CH2, CH3 and optionally CH4). Similarly, from N- to C-terminus, each light chain has a variable domain (VL), also called a variable light domain or a light chain variable domain, followed by a constant light chain (CL) domain. The light chain of an antibody may be assigned to one of two types, called kappa (κ) (SEQ ID NO: 16) and lambda (2) (SEQ ID NO: 17), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “anti-viral cytokine” denotes a cytokines that mediates the establishment of an anti-viral response after infection and recruits inflammatory cells to the site of infection. Anti-viral cytokines comprise type I (interferon(IFN)-α and IFN-β), type II (IFN-γ) and type III (IFN-λ or interleukin(IL)-28/29) interferon. Interferon α, β, γ and λ are important interferons produced in the innate immune response to viral infections.

The term “chimeric” antibody denotes an antibody in which a portion of the heavy and/or light chain is derived from a particular source or species, while the remainder of the heavy and/or light chain is derived from a different source or species. In certain embodiments a chimeric antibody comprises variable domains derived from a first source or species, while the remainder of the heavy and light chain is derived from a second different source or species.

The “class” of an antibody refers to the type of constant domain or constant region possessed by its heavy chain. There are five major classes of human antibodies: IgA, IgD, IgE, IgG, and IgM, and several of these may be further divided into subclasses (isotypes), e.g., IgG₁ (SEQ ID NO: 18 and 19), IgG₂, IgG₃, IgG₄ (SEQ ID NO: 21), IgA₁, and IgA₂. The heavy chain constant domains that correspond to the different classes of immunoglobulins are called α, δ, ε, γ, and μ, respectively.

“Effector functions” denotes those biological activities attributable to the Fc-region of an antibody, which vary with the antibody isotype. Examples of antibody effector functions include: C1q binding and complement dependent cytotoxicity (CDC), Fc receptor binding (FcRn), antibody-dependent cell-mediated cytotoxicity (ADCC), antibody-dependent macrophage-mediated cytotoxicity (ADMC), down regulation of cell surface receptors (e.g. B-cell receptor), and B-cell activation.

An “effective amount” of an agent, e.g., a pharmaceutical formulation, denotes an amount effective, at dosages and for periods of time necessary, to achieve the desired therapeutic or prophylactic result or effect.

The term “Fc-region” denotes the C-terminal region of an immunoglobulin heavy chain that contains at least a portion of the constant region. The term includes native sequence Fc-regions and Fc-regions variants. In one embodiment, a human IgG heavy chain Fc-region extends from about amino acid residue 226 (Cys), or from about amino acid residue 230 (Pro), to the carboxy-terminus of the heavy chain. However, the C-terminal lysine residue (Lys447) of the Fc-region may or may not be present. Unless otherwise specified herein, numbering of amino acid residues of antibody light and heavy chains is according to the EU numbering system, also called the EU index, as described in Kabat et al., Sequences of Proteins of Immunological Interest, 5th ed., Vols. 1-3, Public Health Service, National Institutes of Health, Publication No. 91-3242, Bethesda, Md. (1991).

The term “constant region derived from human origin” denotes a constant heavy chain region of a human antibody of the subclass IgG1, IgG2, IgG3, or IgG4 (comprising e.g. the CH1 domain, the hinge region, the CH2 domain, the CH3 domain, and optionally the CH4 domain) and/or a constant light chain K or 2 region (the CL domain). Such constant regions are well known in the state of the art and e.g. described by Kabat, E. A. (see e.g. Johnson, G., and Wu, T. T., Nucleic Acids Res. 28 (2000) 214-218; Kabat, E. A., et al., Proc. Natl. Acad. Sci. USA 72 (1975) 2785-2788). While antibodies of the IgG4 subclass show reduced Fc receptor (FcγRIIIa) binding, antibodies of other IgG subclasses show strong binding. However Pro238, Asp265, Asp270, Asn 297 (loss of Fc carbohydrate), Pro329, Leu234, Leu235, Gly236, Gly237, Ile253, Ser254, Lys288, Thr307, Gln311, Asn434, and His435 are residues which, if altered, provide also reduced Fc receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment the antibody of the fusion protein has a constant region derived from human origin. In another embodiment the antibody of the fusion protein has a constant region with an amino acid sequence selected from SEQ ID NO: 18 to SEQ ID NO: 22. In also an embodiment the antibody of the fusion protein has a constant region that has the amino acid sequence of SEQ ID NO: 18 or 19.

“Framework” or “FR” denotes variable domain residues other than hypervariable region (HVR) residues or complementarity determining region (CDR) residues. The FR of a variable domain generally consists of four FR domains: FR1, FR2, FR3, and FR4. Accordingly, the HVR (CDR) and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to denote an antibody having a structure substantially similar to a native antibody structure or having heavy chains that contain an Fc-region as defined herein.

The terms “host cell,” “host cell line,” and “host cell culture” are used interchangeably and refer to cells into which exogenous nucleic acid has been introduced, including the progeny of such cells. Host cells include “transformants”, “transformed cells” and “transfected cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th ed., Public Health Service, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

The term “humanized antibody” refers to a chimeric antibody comprising amino acid residues from non-human HVRs, especially CDRs, and amino acid residues from human FRs. In certain embodiments, a humanized antibody will comprise substantially all of at least one, and typically two, variable domains, in which all or substantially all of the HVRs (CDRs) correspond to those of a non-human antibody, and all or substantially all of the FRs correspond to those of a human antibody. A humanized antibody optionally may comprise at least a portion of an antibody constant region derived from human origin. A “humanized variant” of an antibody, e.g., a non-human antibody, refers to an antibody that has undergone humanization. A humanized antibody or a humanized variant of an antibody may comprise amino acid changes in the FRs and the constant region.

The term “hypervariable region” or “HVR” as used herein refers to each of the regions of an antibody variable domain which are hypervariable in sequence and/or form structurally defined loops (“hypervariable loops”). Generally, native four-chain antibodies comprise six HVRs, whereof three are in the VH (H1, H2, H3), and three in the VL (L1, L2, L3). HVRs generally comprise amino acid residues from the hypervariable loops or from the “complementarity determining regions” (CDRs), being of highest sequence variability and/or involved in antigen recognition. Hypervariable loops occur in one embodiment at amino acid residues 26-32 (L1), 50-52 (L2), 91-96 (L3) of the VL domain and 26-32 (H1), 53-55 (H2), and 96-101 (H3) of the VH domain (Chothia and Lesk, J. Mol. Biol. 196 (1987) 901-917). CDRs (CDR-L1, CDR-L2, CDR-L3, CDR-H1, CDR-H2, and CDR-H3) occur in one embodiment at amino acid residues 24-34 (L1), 50-56 (L2), 89-97 (L3) for the VL domain and 31-35B (H1), 50-65 (H2), and 95-102 (H3) of the VH domain (Kabat et al., Sequences of Proteins of Immunological Interest, 5th ed., vols. 1-3, Public Health Service, National Institutes of Health, Publication No. 91-3242, Bethesda, Md. (1991)). With the exception of CDR1 in VH, CDRs generally comprise the amino acid residues that form the hypervariable loops. CDRs also comprise “specificity determining residues”, or “SDRs”, which are residues that contact the antigen. SDRs are contained within regions of the CDRs called abbreviated-CDRs, or a-CDRs. a-CDRs (a-CDR-L1, a-CDR-L2, a-CDR-L3, a-CDR-H1, a-CDR-H2, and a-CDR-H3) occur in one embodiment at amino acid residues 31-34 (L1), 50-55 (L2), 89-96 (L3) of the VL domain and 31-35B (H1), 50-58 (H2), and 95-102 (H3) of the VH domain (see e.g. Almagro, J. C. and Fransson, J., Front. Biosci. 13 (2008) 1619-1633). Unless otherwise indicated, HVR residues and other residues in the variable domain (e.g., FR residues) are numbered herein according to Kabat et al., supra. An “immunoconjugate” is an antibody conjugated to one or more heterologous molecule(s), including but not limited to a cytotoxic agent.

An “individual” or “subject” is a mammal. Mammals include, but are not limited to, primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman, S., et al., J. Chromatogr. B 848 (2007) 79-87.

An “isolated” nucleic acid refers to a nucleic acid molecule that has been separated from a component of its natural environment. (An isolated nucleic acid includes a nucleic acid molecule contained in cells that ordinarily contain the nucleic acid molecule, but the nucleic acid molecule is present extrachromosomally or at a chromosomal location that is different from its natural chromosomal location.)

“Isolated nucleic acid encoding an antibody that binds to a human major histocompatibility complex presenting a peptidic fragment of an hepatitis-B-virus protein” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, single antibody producing cell isolation methods, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are hetero-tetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three or four constant domains (CH1, CH2, CH3 and optionally CH4). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “type I interferon” denotes interferons that bind to the cell surface receptor complex which consists of IFNAR1 and IFNAR2 protein chains (the IFN-α receptor, IFNAR). The type I interferons present in humans comprise interferon α, interferon β and interferon ω.

The term “type II interferon” denotes interferons that bind to the interferon-gamma receptor (IFNGR). The type II interferons present in humans comprise interferon γ.

The term “type III interferon” denotes interferons that signal through a receptor complex consisting of class II cytokine receptor (CIICR) IL10R2 and IFNLR1. The type III interferon group consists of 3 IFN-λ molecules called IFN-λ1, IFN-λ2 and IFN-λ3 (also called interleukin-29, interleukin-28A and interleukin-28B, respectively).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt et al., Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano, S. et al., J. Immunol. 150 (1993) 880-887; Clarkson, T., et al., Nature 352 (1991) 624-628).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

The fusion proteins as reported herein demonstrated sensitivity similar to HBV-specific CD8 T cells from resolved hepatitis patients. They also recognize ex vivo HBV-infected hepatocytes from chronic HBV patients. This recognition was not affected by the presence of circulating HBV antigens. Importantly, the fusion of the antibody to interferon-alpha did not alter the sensitivity of the antibody to cells expressing HBV antigens, while the affinity of the fused interferon-alpha to its own receptor was reduced. It has been found that interferon-alpha activity was markedly enhanced on cells expressing HBV antigens. Pre-blocking of the MHC/peptide sites with TCRL abrogated the enhanced interferon-alpha activity of the fusion protein as reported herein (FIG. 9).

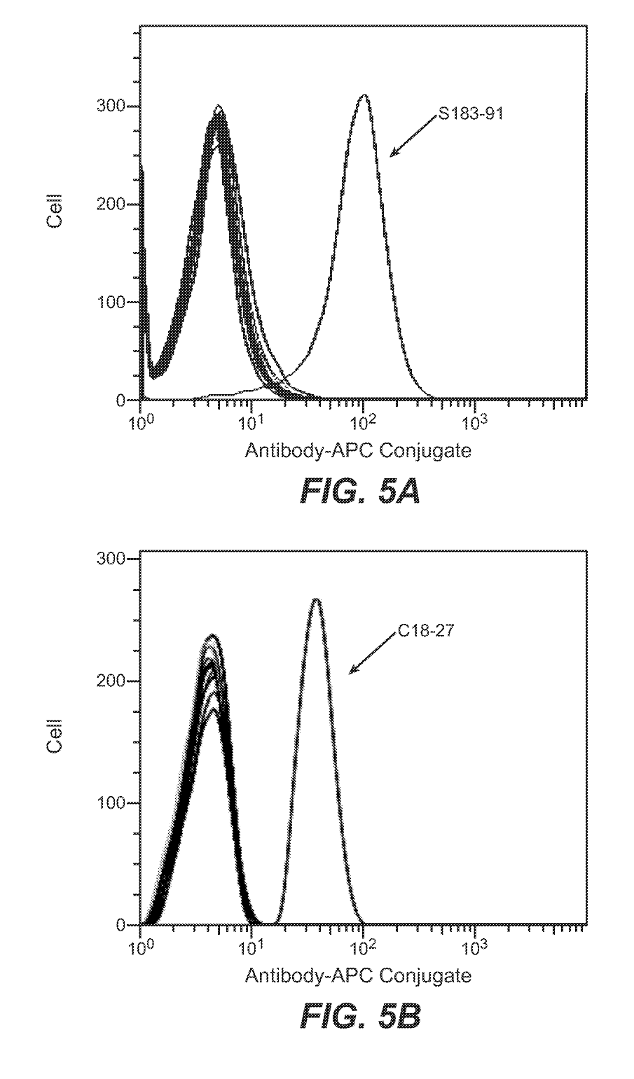

The specificity of the antibodies to HBV infected cells is shown in FIG. 5. In FIG. 5(A) only the antibody that binds to a human major histocompatibility complex presenting the peptidic fragment of SEQ ID NO: 31 of a hepatitis-B-virus protein shows binding; in FIG. 5(B) only the antibody that binds to a human major histocompatibility complex presenting the peptidic fragment of SEQ ID NO: 30 of a hepatitis-B-virus protein shows binding.

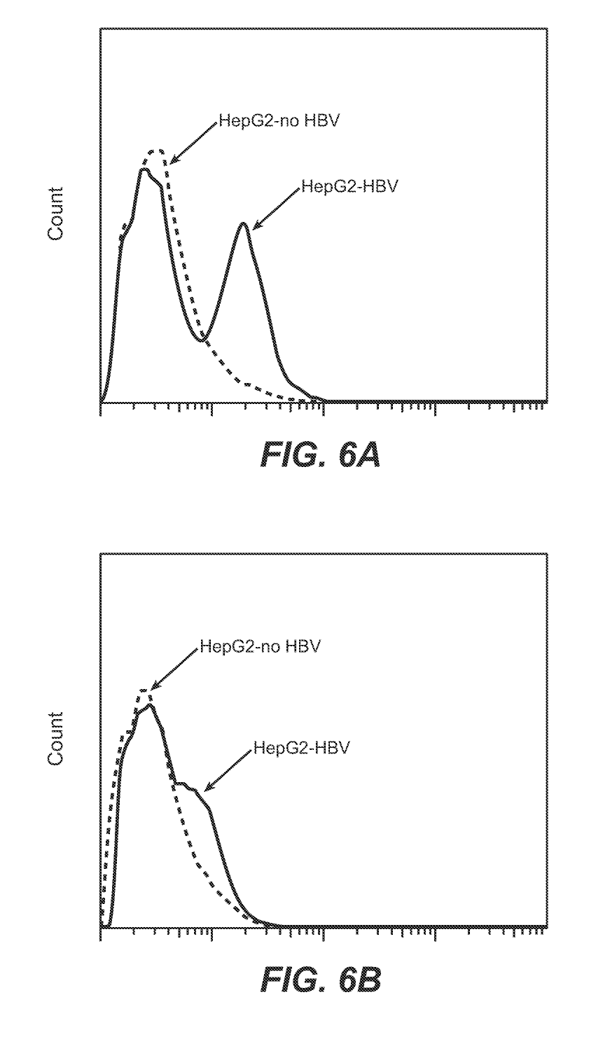

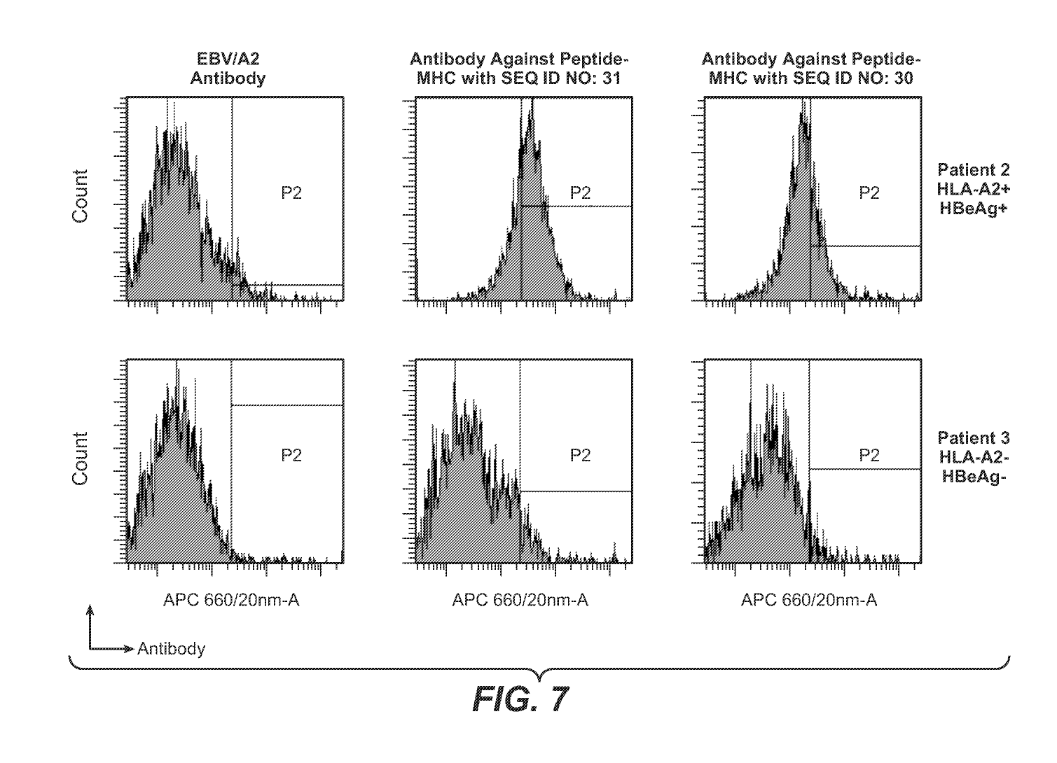

The recognition of peptide-MHC complexes on infected hepatocytes is shown in FIGS. 6 and 7.

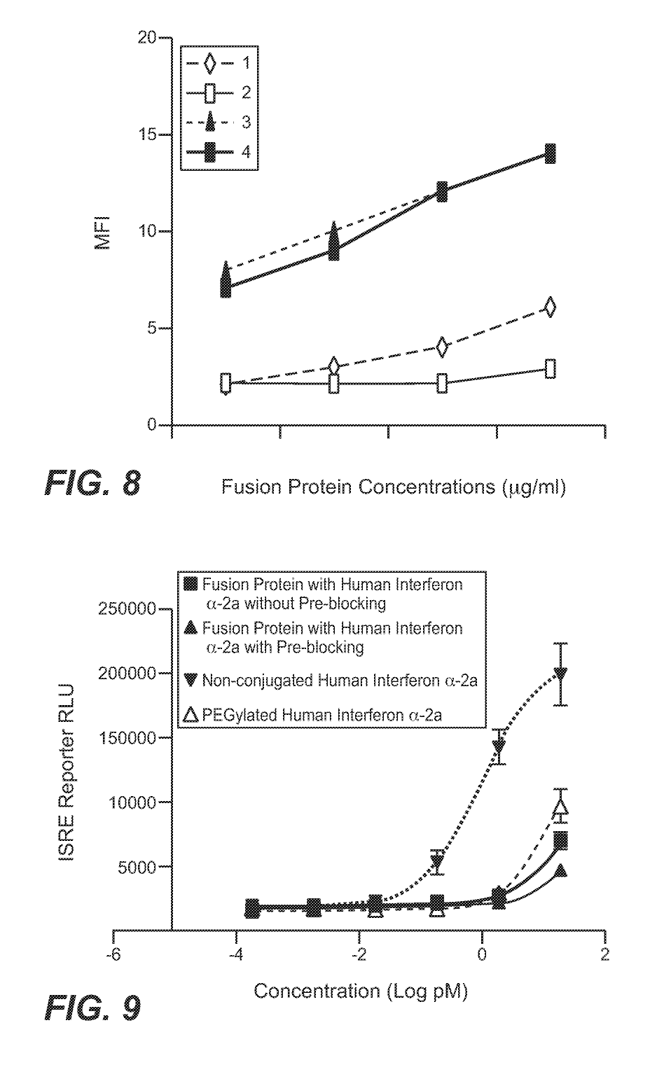

FIG. 8 shows that the fusion protein as reported herein maintains the specificity of the non-conjugated antibody that binds to a human major histocompatibility complex presenting a peptidic fragment of a hepatitis-B-virus protein.

In one aspect, the invention is based, in part, on the development of a fusion protein comprising an antibody that binds to a human major histocompatibility complex presenting a peptidic fragment of an hepatitis-B-virus protein and a anti-viral cytokine, which is e.g. for delivering an anti-viral cytokine to hepatitis-B-virus infected hepatocytes. The fusion proteins of the invention are useful, e.g., for the treatment of subjects infected with hepatitis-B-virus.

In one aspect are reported fusion proteins comprising an antibody with specificity for the peptide/MHC-I of HBV envelope (envelope 183-191/A201) and HBV core (core 18-27/A201) antigens presented on HBV infected cells. The antibody mimics T-cell receptor recognition of HBV-specific CD8 T-cells.

A. Exemplary Fusion Protein Comprising an Antibody that Binds to a Human Major Histocompatibility Complex Presenting a Peptidic Fragment of an Hepatitis-B-Virus Protein and an Anti-Viral Cytokine

In one aspect, the invention provides a fusion protein comprising an antibody that binds to a human major histocompatibility complex presenting a peptidic fragment of a hepatitis-B-virus protein and an anti-viral cytokine.

In one aspect, the invention provides a fusion protein comprising an antibody that binds to a human major histocompatibility complex presenting a peptidic fragment of an hepatitis-B-virus protein comprising at least one, two, three, four, five, or six HVRs selected from (a) HVR-H1 comprising the amino acid sequence of SEQ ID NO: 04, (b) HVR-H2 comprising the amino acid sequence of SEQ ID NO: 05, (c) HVR-H3 comprising the amino acid sequence of SEQ ID NO: 06, (d) HVR-L1 comprising the amino acid sequence of SEQ ID NO: 08, (e) HVR-L2 comprising the amino acid sequence of SEQ ID NO: 09, and (f) HVR-L3 comprising the amino acid sequence of SEQ ID NO: 10.

In one aspect, the invention provides a fusion protein comprising an antibody that binds to a human major histocompatibility complex presenting a peptidic fragment of an hepatitis-B-virus protein comprising at least one, two, three, four, five, or six HVRs selected from (a) HVR-H1 comprising the amino acid sequence of SEQ ID NO: 32, (b) HVR-H2 comprising the amino acid sequence of SEQ ID NO: 33, (c) HVR-H3 comprising the amino acid sequence of SEQ ID NO: 34, (d) HVR-L1 comprising the amino acid sequence of SEQ ID NO: 36, (e) HVR-L2 comprising the amino acid sequence of SEQ ID NO: 37, and (f) HVR-L3 comprising the amino acid sequence of SEQ ID NO: 38.

In one aspect, the invention provides a fusion protein comprising an antibody that binds to a human major histocompatibility complex presenting a peptidic fragment of an hepatitis-B-virus protein comprising at least one, at least two, or all three VH HVR sequences selected from (a) HVR-H1 comprising the amino acid sequence of SEQ ID NO: 04, (b) HVR-H2 comprising the amino acid sequence of SEQ ID NO: 05, and (c) HVR-H3 comprising the amino acid sequence of SEQ ID NO: 06. In one embodiment, the antibody comprises a HVR-H3 comprising the amino acid sequence of SEQ ID NO: 06. In one embodiment, the antibody comprises HVR-H3 comprising the amino acid sequence of SEQ ID NO: 06 and HVR-L3 comprising the amino acid sequence of SEQ ID NO: 10. In one embodiment, the antibody comprises HVR-H3 comprising the amino acid sequence of SEQ ID NO: 06, HVR-L3 comprising the amino acid sequence of SEQ ID NO: 10, and HVR-H2 comprising the amino acid sequence of SEQ ID NO: 05.

In one aspect, the invention provides a fusion protein comprising an antibody that binds to a human major histocompatibility complex presenting a peptidic fragment of an hepatitis-B-virus protein comprising at least one, at least two, or all three VH HVR sequences selected from (a) HVR-H1 comprising the amino acid sequence of SEQ ID NO: 32, (b) HVR-H2 comprising the amino acid sequence of SEQ ID NO: 33, and (c) HVR-H3 comprising the amino acid sequence of SEQ ID NO: 34. In one embodiment, the antibody comprises a HVR-H3 comprising the amino acid sequence of SEQ ID NO: 34. In one embodiment, the antibody comprises HVR-H3 comprising the amino acid sequence of SEQ ID NO: 34 and HVR-L3 comprising the amino acid sequence of SEQ ID NO: 38. In one embodiment, the antibody comprises HVR-H3 comprising the amino acid sequence of SEQ ID NO: 34, HVR-L3 comprising the amino acid sequence of SEQ ID NO: 38, and HVR-H2 comprising the amino acid sequence of SEQ ID NO: 33.

In one aspect, the invention provides a fusion protein comprising an antibody which comprises at least one, at least two, or all three VL HVR sequences selected from (a) HVR-L1 comprising the amino acid sequence of SEQ ID NO: 08, (b) HVR-L2 comprising the amino acid sequence of SEQ ID NO: 09, and (c) HVR-L3 comprising the amino acid sequence of SEQ ID NO: 10.

In one aspect, the invention provides a fusion protein comprising an antibody which comprises at least one, at least two, or all three VL HVR sequences selected from (a) HVR-L1 comprising the amino acid sequence of SEQ ID NO: 36, (b) HVR-L2 comprising the amino acid sequence of SEQ ID NO: 37, and (c) HVR-L3 comprising the amino acid sequence of SEQ ID NO: 38.

In one aspect, a fusion protein of the invention comprises an antibody with (a) a VH domain comprising at least one, at least two, or all three VH HVR sequences selected from (i) HVR-H1 comprising the amino acid sequence of SEQ ID NO: 04, (ii) HVR-H2 comprising the amino acid sequence of SEQ ID NO: 05, and (iii) HVR-H3 comprising an amino acid sequence selected from SEQ ID NO: 06, and (b) a VL domain comprising at least one, at least two, or all three VL HVR sequences selected from (i) HVR-L1 comprising the amino acid sequence of SEQ ID NO: 08, (ii) HVR-L2 comprising the amino acid sequence of SEQ ID NO: 09, and (c) HVR-L3 comprising the amino acid sequence of SEQ ID NO: 10.

In one aspect, a fusion protein of the invention comprises an antibody with (a) a VH domain comprising at least one, at least two, or all three VH HVR sequences selected from (i) HVR-H1 comprising the amino acid sequence of SEQ ID NO: 32, (ii) HVR-H2 comprising the amino acid sequence of SEQ ID NO: 33, and (iii) HVR-H3 comprising an amino acid sequence selected from SEQ ID NO: 34, and (b) a VL domain comprising at least one, at least two, or all three VL HVR sequences selected from (i) HVR-L1 comprising the amino acid sequence of SEQ ID NO: 36, (ii) HVR-L2 comprising the amino acid sequence of SEQ ID NO: 37, and (c) HVR-L3 comprising the amino acid sequence of SEQ ID NO: 38.

In one aspect, the fusion protein comprising an antibody that binds to a human major histocompatibility complex presenting a peptidic fragment of an hepatitis-B-virus protein and a anti-viral cytokine comprises an antibody that binds to a human major histocompatibility complex presenting a peptidic fragment of an hepatitis-B-virus protein that comprises a heavy chain variable domain (VH) amino acid sequence having at least 90%, 91%, 92%, 93%, 94%, 95%, 96%, 97%, 98%, 99%, or 100% sequence identity to the amino acid sequence of SEQ ID NO: 07, or SEQ ID NO: 35, or to a humanized variant thereof. In certain embodiments, a VH amino acid sequence having at least 90%, 91%, 92%, 93%, 94%, 95%, 96%, 97%, 98%, or 99% identity contains substitutions (e.g. conservative substitutions), insertions, or deletions relative to the reference sequence, but retains the ability to bind to a human major histocompatibility complex presenting a peptidic fragment of an hepatitis-B-virus protein. In a particular embodiment, the VH comprises one, two or three HVRs selected from: (a) HVR-H1 comprising the amino acid sequence of SEQ ID NO: 04, (b) HVR-H2 comprising the amino acid sequence of SEQ ID NO: 05, and (c) HVR-H3 comprising the amino acid sequence of SEQ ID NO: 06. In a particular embodiment, the VH comprises one, two or three HVRs selected from: (a) HVR-H1 comprising the amino acid sequence of SEQ ID NO: 32, (b) HVR-H2 comprising the amino acid sequence of SEQ ID NO: 33, and (c) HVR-H3 comprising the amino acid sequence of SEQ ID NO: 34.

In one aspect, the fusion protein comprising an antibody that binds to a human major histocompatibility complex presenting a peptidic fragment of an hepatitis-B-virus protein and a anti-viral cytokine comprises an antibody that binds to a human major histocompatibility complex presenting a peptidic fragment of an hepatitis-B-virus protein comprising a light chain variable domain (VL) having at least 90%, 91%, 92%, 93%, 94%, 95%, 96%, 97%, 98%, 99%, or 100% sequence identity to the amino acid sequence of SEQ ID NO: 11, or SEQ ID NO: 39, or to a humanized variant thereof. In certain embodiments, a VL sequence having at least 90%, 91%, 92%, 93%, 94%, 95%, 96%, 97%, 98%, or 99% identity contains substitutions (e.g. conservative substitutions), insertions, or deletions relative to the reference sequence, but retains the ability to bind to a human major histocompatibility complex presenting a peptidic fragment of an hepatitis-B-virus protein. In another particular embodiment, the VL comprises one, two or three HVRs selected from (a) HVR-L1 comprising the amino acid sequence of SEQ ID NO: 08, (b) HVR-L2 comprising the amino acid sequence of SEQ ID NO: 09, and (c) HVR-L3 comprising the amino acid sequence of SEQ ID NO: 10. In another particular embodiment, the VL comprises one, two or three HVRs selected from (a) HVR-L1 comprising the amino acid sequence of SEQ ID NO: 36, (b) HVR-L2 comprising the amino acid sequence of SEQ ID NO: 37, and (c) HVR-L3 comprising the amino acid sequence of SEQ ID NO: 38.

In one aspect, a fusion protein comprising an antibody that binds to a human major histocompatibility complex presenting a peptidic fragment of an hepatitis-B-virus protein and a anti-viral cytokine comprising an antibody that binds to a human major histocompatibility complex presenting a peptidic fragment of an hepatitis-B-virus protein is provided, wherein the antibody comprises a VH as in any of the embodiments provided above, and a VL as in any of the embodiments provided above. In one embodiment, the antibody comprises the VH and VL sequences in SEQ ID NO: 07 and SEQ ID NO: 11, respectively, including post-translational modifications of those sequences, or humanized variants thereof. In one embodiment, the antibody comprises the VH and VL sequences in SEQ ID NO: 35 and SEQ ID NO: 39, respectively, including post-translational modifications of those sequences, or humanized variants thereof.

In one aspect, the invention provides a fusion protein comprising an antibody that binds to the same epitope as an antibody that binds to a human major histocompatibility complex presenting a peptidic fragment of an hepatitis-B-virus protein with a VH of SEQ ID NO: 07 and a VL of SEQ ID NO: 11.

In one aspect, the invention provides a fusion protein comprising an antibody that binds to the same epitope as an antibody that binds to a human major histocompatibility complex presenting a peptidic fragment of an hepatitis-B-virus protein with a VH of SEQ ID NO: 35 and a VL of SEQ ID NO: 39.

In one aspect of the invention, the antibody of the fusion protein according to any of the above embodiments and aspects is a monoclonal antibody, including a chimeric, humanized, or human antibody. In one embodiment, the antibody is an antibody fragment, e.g., a Fv, Fab, Fab′, scFv, diabody, or F(ab′)₂ fragment. In one embodiment, the antibody is a full length antibody, e.g., an intact IgG1 antibody or other antibody class or isotype as defined herein.

In one aspect, a fusion protein according to any of the above embodiments and aspects may incorporate any of the features, singly or in combination, as described in the sections below:

1. Affinity

In certain embodiments, a fusion protein as provided herein or the antibody comprised in the fusion protein as provided herein has a dissociation constant (Kd) of ≦10 nM, ≦1 nM, ≦0.1 nM, ≦0.01 nM, or ≦0.001 nM (e.g. 10⁻⁸M or less, e.g. from 10⁻⁸ M to 10⁻¹³ M, e.g., from 10⁻⁹ M to 10⁻¹³ M) from a human major histocompatibility complex presenting a peptidic fragment of a hepatitis-B-virus protein.

In one embodiment, Kd is measured by a surface plasmon resonance method.

Binding affinities of interferon α-2a or of fusions containing interferon α-2a towards the human interferon-alpha/beta receptor beta chain (IFNAR2) can be determined by Surface Plasmon Resonance (SPR) using a BIAcore® 3000 instrument (GE Healthcare) at 25° C. IFNAR2 is the high-affinity, initial binding component of the heterodimeric interferon receptor complex consisting out of IFNAR1/2 and interferon α-2a as Ligand.

The BIAcore® system is well established for the study of molecule interactions. It allows a continuous real-time monitoring of ligand/analyte bindings and, thus, the determination of association rate constants (ka), dissociation rate constants (kd), and equilibrium dissociation constants (Kd). SPR-technology is based on the measurement of the refractive index close to the surface of a gold coated biosensor chip. Changes in the refractive index indicate mass changes on the surface caused by the interaction of immobilized ligand with analyte injected in solution. If molecules bind immobilized ligand on the surface the mass increases, in case of dissociation the mass decreases.

Amine coupling of around 750 resonance units (RU) of a capturing system (e.g. capturing monoclonal antibody specifically binding to human IgG, Jackson Immunoresearch) can be performed on a CM5 chip at pH 4.5 using an amine coupling kit supplied by GE Healthcare. huFc-tagged IFNAR2 (RnD Systems, Cat-Nr. 4015-AB) can be captured at a concentration of 5 μg/ml. Excess binding sites can be blocked by injecting a human Fc-part (huFc) mixture at a concentration of 1.25 μM (Biodesign, Cat-Nr. 50175). Different concentrations of interferon or interferon fusion proteins ranging from 0.1 nM to 50 nM can be passed with a flow rate of 10 μL1/min through the flow cells at 298 K for 120-240 sec. to record the association phase. The dissociation phase can be monitored for up to 600 sec. and can be triggered by switching from the sample solution to running buffer. The surface can be regenerated by 1 min washing with a 100 mM phosphoric acid solution at a flow rate of 30 μl/min. For the experiments a HBS-P+buffer supplied by GE Healthcare can be chosen (10 mM HEPES, pH 7.4, 150 mM NaCl, 0.05% (v/v) Surfactant P20).

Bulk refractive index differences can be corrected for by subtracting the response obtained from a blank-coupled surface. Blank injections are also substracted (=double referencing).

The equilibrium dissociation constant (Kd), defined as ka/kd, can be determined by analyzing the sensogram curves obtained with several different concentrations, using BIAevaluation 4.1 software package. The fitting of the data followed a suitable binding model.

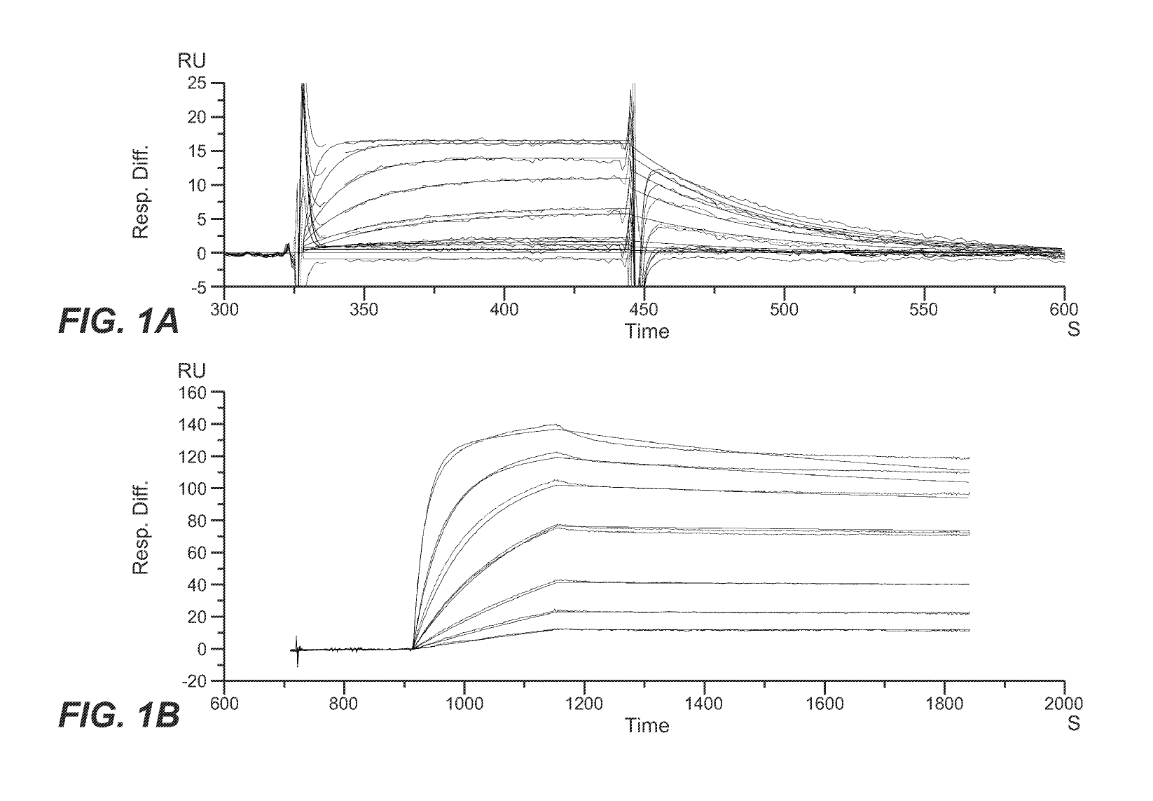

For the determination of the Kd of human wildtype interferon α-2a 0.1 nM to 50 nM interferon α-2a can be injected over an IFNAR2 coated sensor chip. A corresponding sensogram is shown in FIG. 1 a). For human interferon α-2a fused C-terminally to an Fc-region of human origin, such a fusion protein can be injected at a concentration of 0.5 nM to 50 nM over an IFNAR2 coated surface. Complex stability increases from 35 sec. for interferon α-2a to 23 min. for an interferon α-2a Fc-part-fusion protein. Respectively, the affinity increases from 4 nM for interferon α-2a to an apparent affinity of 0.3 nM for the fusion protein. Since for activity IFNAR1 is essential only initial binding can be addressed. No interferon signaling activity can be addressed by such an assay. In one embodiment the fusion protein has a binding affinity for IFNAR2 of 1 nM or less.

2. Antibody Fragments

In certain embodiments, the antibody of the fusion protein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson, P. J., et al., Nat. Med. 9 (2003) 129-134. For a review of scFv fragments, see, e.g., Plueckthun, In: The Pharmacology of Monoclonal Antibodies, Vol. 113, Rosenburg and Moore (eds.), Springer-Verlag, New York, pp. 269-315 (1994); WO 93/16185; U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 0 404 097; WO 1993/01161; Hudson, P. J., et al., Nat. Med. 9 (2003) 129-134; Hollinger, P. et al., Proc. Natl. Acad. Sci. USA 90 (1993) 6444-6448. Triabodies and tetrabodies are also described in Hudson, P. J., et al., Nat. Med. 9 (2003) 129-134.

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516).

Antibody fragments can be made by various techniques, including but not limited to production by recombinant host cells (e.g. E. coli or phage), as described herein.

3. Chimeric and Humanized Antibodies

In certain embodiments, the antibody of the fusion protein is a chimeric antibody. Certain chimeric antibodies are reported, e.g., in U.S. Pat. No. 4,816,567; and Morrison, L. E., et al., Proc. Natl. Acad. Sci. USA 81 (1984) 6851-6855. In one example, a chimeric antibody comprises a non-human variable region (i.e., a variable region derived from mouse) and a constant region of human origin. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a constant region of human origin. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C. and Fransson, J., Front. Biosci. 13 (2008) 1619-1633, and are further reported, e.g., in Riechmann, L., et al., Nature 332 (1988) 323-327; Queen, C., et al., Proc. Natl. Acad. Sci. USA 86 (1989) 10029-10033; U.S. Pat. No. 5,821,337, U.S. Pat. No. 7,527,791, U.S. Pat. No. 6,982,321, and U.S. Pat. No. 7,087,409; Kashmiri, S. V., et al., Methods 36 (2005) 25-34 (reporting SDR (a-CDR) grafting); Padlan, Mol. Immunol. 28 (1991) 489-498 (reporting “resurfacing”); Dall'Acqua, W. F., et al., Methods 36 (2005) 43-60 (reporting “FR shuffling”); and Osbourn, J., et al., Methods 36 (2005) 61-68 and Klimka, A., et al., Br. J. Cancer 83 (2000) 252-260 (reporting the “guided selection” approach to FR shuffling).

Human framework regions that may be used for humanization include but are not limited to: framework regions selected using the “best-fit” method (see, e.g., Sims, J. E., et al., J. Immunol. 151 (1993) 2296-2308), framework regions derived from the consensus sequence of human antibodies of a particular subgroup of light or heavy chain variable regions (see, e.g., Carter, P., et al., Proc. Natl. Acad. Sci. USA, 89 (1992) 4285-4289; Presta, L. G., et al., J. Immunol. 151 (1993) 2623-2632), human mature (somatically mutated) framework regions or human germline framework regions (see, e.g., Almagro, J. C. and Fransson, J., Front. Biosci. 13 (2008) 1619-1633), and framework regions derived from screening FR libraries (see, e.g., Baca, M., et al., J. Biol. Chem. 272 (1997) 10678-10684; Rosok, M. J., et al., J. Biol. Chem. 271 (1996) 22611-22618).

4. Human Antibodies

In certain embodiments, the antibody of the fusion protein is a human antibody. Human antibodies can be produced using various techniques known in the art. Human antibodies are described generally in van Dijk, M. A. and van de Winkel, J. G., Curr. Opin. Chem. Biol. 5 (2001) 368-374; Lonberg, N., Curr. Opin. Immunol. 20 (2008) 450-459.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, N., Nat. Biotech. 23 (2005) 1117-1125. See also, e.g., U.S. Pat. No. 6,075,181 and U.S. Pat. No. 6,150,584 reporting XENOMOUSE™ technology; U.S. Pat. No. 5,770,429 reporting HuMAB® technology; U.S. Pat. No. 7,041,870 reporting K-M MOUSE® technology; and US 2007/0061900 reporting VELOCIMOUSE® technology). Human variable regions from intact antibodies generated by such animals may be further modified, e.g., by combining with a different human constant region.

Human antibodies can also be made by hybridoma-based methods. Human myeloma and murine-human heteromyeloma cell lines for the production of human monoclonal antibodies have been reported (see, e.g., Kozbor, D., J. Immunol. 133 (1984) 3001-3005; Brodeur et al., Monoclonal Antibody Production Techniques and Applications, Marcel Dekker, Inc., New York (1987) pp. 51-63; Boerner, P., et al., J. Immunol. 147 (1991) 86-95). Human antibodies generated via human B-cell hybridoma technology are also described in L1, J., et al., Proc. Natl. Acad. Sci. USA 103 (2006) 3557-3562. Additional methods include those described, for example, in U.S. Pat. No. 7,189,826 (reporting production of monoclonal human IgM antibodies from hybridoma cell lines) and Ni, J., Xiandai Mianyixue 26 (2006) 265-268 (reporting human-human hybridomas). Human hybridoma technology (Trioma technology) is also reported in Vollmers, H. P. and Brandlein, S., Histology and Histopathology 20 (2005) 927-937; Vollmers, H. P. and Brandlein, S., Methods and Findings in Experimental and Clinical Pharmacology 27 (2005) 185-191.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies comprised in the fusion protein of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom, H. R., et al., Methods in Molecular Biology 178 (2001) 1-37 (O'Brien et al., ed., Human Press, Totowa, N.J.) and further reported, e.g., in McCafferty, J. et al., Nature 348 (1990) 552-554; Clackson, T. et al., Nature 352 (1991) 624-628; Marks, J. D. et al., J. Mol. Biol. 222 (1991) 581-597; Marks, J. D. et al., in Methods in Molecular Biology 248 (2003) 161-176; Sidhu, S. S. et al., J. Mol. Biol. 338 (2004) 299-310; Lee, C. V., et al., J. Mol. Biol. 340 (2004) 1073-1093; Fellouse, F. A., Proc. Natl. Acad. Sci. USA 101 (2004) 12467-12472; Lee, C. V. et al., J. Immunol. Methods 284 (2004) 119-132.

In certain phage display methods, repertoires of VH and VL genes are separately cloned by polymerase chain reaction (PCR) and recombined randomly in phage libraries, which can then be screened for antigen-binding phage as described in Winter, G. et al., Ann. Rev. Immunol. 12 (1994) 433-455. Phages typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths, A. D. et al., EMBO J. 12 (1993) 725-734. Finally, naive libraries can also be made synthetically by cloning non-rearranged V-gene segments from stem cells, and using PCR primers containing random sequence to encode the highly variable CDR3 regions and to accomplish rearrangement in vitro, as reported by Hoogenboom, H. R. and Winter, G., J. Mol. Biol. 227 (1992) 381-388. Patent publications reporting human antibody phage libraries include, for example, U.S. Pat. No. 5,750,373, US 2005/0079574, US 2005/0119455, US 2005/0266000, US 2007/0117126, US 2007/0160598, US 2007/0237764, US 2007/0292936, and US 2009/0002360.

Antibodies or antibody fragments isolated from human antibody libraries are considered human antibodies or human antibody fragments herein.

6. Multispecific Antibodies

In certain embodiments, the fusion protein as reported herein comprises an antibody which is a multispecific antibody, e.g. a bispecific antibody. Multispecific antibodies are monoclonal antibodies that have binding specificities for at least two different sites. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540); WO 93/08829; and Traunecker, A. et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980; and Brennan, M. et al., Science 229 (1985) 81-83); using leucine zippers to produce bi-specific antibodies (see, e.g., Kostelny, S. A. et al., J. Immunol. 148 (1992) 1547-1553); using “diabody” technology for making bispecific antibody fragments (see, e.g., Hollinger, P. et al., Proc. Natl. Acad. Sci. USA 90 (1993) 6444-6448); and using single-chain Fv (sFv) dimers (see, e.g., Gruber, M. et al., J. Immunol. 152 (1994) 5368-5374); and preparing trispecific antibodies as described, e.g., in Tutt, A. et al., J. Immunol. 147 (1991) 60-69.

Engineered antibodies with three or more functional antigen binding sites, including “Octopus antibodies,” are also included herein (see, e.g. US 2006/0025576).

The antibody or fragment also includes a “Dual Acting Fab” or “DAF” comprising an antigen binding site that binds to a first antigen as well as another, different antigen (see, US 2008/0069820, for example).

The antibody or antibody fragment also include multispecific antibodies described in WO 2009/080251, WO 2009/080252, WO 2009/080253, WO 2009/080254, WO 2010/112193, WO 2010/115589, WO 2010/136172, WO 2010/145792, and WO 2010/145793.

7. Antibody Variants

In certain embodiments, amino acid sequence variants of the antibody comprised in the fusion protein provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, fusion proteins comprising an antibody variant having one or more amino acid substitutions are provided. Sites of interest for substitutional mutagenesis include the HVRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into the antibody and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, He;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots”, i.e., residues encoded by codons that undergo mutation at high frequency during the somatic maturation process (see, e.g., Chowdhury, P. S., Methods Mol. Biol. 207 (2003) 179-196), and/or SDRs (a-CDRs), with the resulting variant VH or VL being tested for binding affinity. Affinity maturation by constructing and reselecting from secondary libraries has been reported, e.g., in Hoogenboom, H. R., et al., Methods in Molecular Biology 178 (2001) 1-37 (O'Brien et al., ed., Human Press, Totowa, N.J.).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind its antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham, B. C. and Wells, J. A., Science 244 (1989) 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue.

### b) Glycosylation Variants

In certain embodiments, the fusion protein provided herein comprises an antibody that is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc-region, the carbohydrate attached thereto may be altered. Native antibodies produced by mammalian cells typically comprise a branched, biantennary oligosaccharide that is generally attached by an N-linkage to Asn297 of the CH2 domain of the Fc-region (see, e.g., Wright, A. et al., TIBTECH 15 (1997) 26-32). The oligosaccharide may include various carbohydrates, e.g., mannose, N-acetyl glucosamine (GlcNAc), galactose, and sialic acid (NANA, Neu5Ac), as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, the antibody has a carbohydrate structure that lacks fucose attached (directly or indirectly) to an Fc-region. For example, the amount of fucose in such antibody may be from 1% to 80%, from 1% to 65%, from 5% to 65%, from 5% to 20% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as reported in WO 2008/077546, for example. Asn297 refers to the asparagine residue located at about position 297 in the Fc-region (Eu numbering of Fc-region residues). However, Asn297 may also be located about ±3 amino acids upstream or downstream of position 297, i.e., between positions 294 and 300, due to minor sequence variations in antibodies. Such fucosylation variants may have improved ADCC function (see, e.g., US 2003/0157108 and US 2004/0093621). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki, A. et al., J. Mol. Biol. 336 (2004) 1239-1249; Yamane-Ohnuki, N. et al., Biotech. Bioeng. 87 (2004) 614-622. Examples of cell lines capable of producing defucosylated antibodies include Lec13 CHO cells deficient in protein fucosylation (Ripka, J. et al., Arch. Biochem. Biophys. 249 (1986) 533-545; US 2003/0157108; WO 2004/056312, especially at Example 11), and knockout cell lines, such as alpha-1,6-fucosyltransferase gene, FUT8, knockout CHO cells (see, e.g., Yamane-Ohnuki, N. et al., Biotech. Bioeng. 87 (2004) 614-622; Kanda, Y. et al., Biotechnol. Bioeng. 94 (2006) 680-688; WO 2003/085107).

Further fusion proteins are provided comprising an antibody with bisected oligosaccharides, e.g., in which a biantennary oligosaccharide attached to the Fc-region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Fusion proteins comprising an antibody with at least one galactose residue in the oligosaccharide attached to the Fc-region are also provided. Such antibody variants may have improved CDC function. Such antibody variants are described, e.g., in WO 1997/30087; WO 1998/58964; and WO 1999/22764.

### c) Fc-Region Variants

In certain embodiments, one or more amino acid modifications may be introduced into the Fc-region of the antibody of the fusion protein provided herein, thereby generating an Fc-region variant. The Fc-region variant may comprise an Fc-region sequence of human origin (e.g., a human IgG1, IgG2, IgG3 or IgG4 Fc-region) comprising an amino acid modification (e.g. a substitution) at one or more amino acid positions.

In certain embodiments, the invention contemplates a fusion protein comprising an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch, J. V. and Kinet, J. P. (Anna. Rev. Immunol. 9 (1991) 457-492). Non-limiting examples of in vitro assays to assess ADCC activity of a molecule of interest is reported in U.S. Pat. No. 5,500,362 (see, e.g., Hellstrom, I. et al., Proc. Natl. Acad. Sci. USA 83 (1986) 7059-7063) and Hellstrom, I. et al., Proc. Natl. Acad. Sci. USA 82 (1985) 1499-1502; U.S. Pat. No. 5,821,337 (see Brueggemann, M. et al., J. Exp. Med. 166 (1987) 1351-1361). Alternatively, non-radioactive assays methods may be employed (see, for example, ACTIT™ non-radioactive cytotoxicity assay for flow cytometry (CellTechnology, Inc. Mountain View, Calif.), and CytoTox 9e non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, R. et al., Proc. Natl. Acad. Sci. USA 95 (1998) 652-656. Clq binding assays may also be carried out to confirm that the antibody is unable to bind Clq and hence lacks CDC activity (see, e.g., Clq and C3c binding ELISA in WO 2006/029879 and WO 2005/100402). To assess complement activation, a CDC assay may be performed (see, for example, Gazzano-Santoro, H., et al., J. Immunol. Methods 202 (1997) 163-171; Cragg, M. S., et al., Blood 101 (2003) 1045-1052; and Cragg, M. S. and M. J. Glennie, Blood 103 (2004) 2738-2743). FcRn binding and in vivo clearance/half life determinations can also be performed using methods known in the art (see, e.g., Petkova, S. B., et al., Int. Immunol. 18 (2006) 1759-1769).

Antibodies with reduced effector function include those with substitution of one or more of Fc-region residues 238, 265, 269, 270, 297, 327 and 329 (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are reported (see, e.g., U.S. Pat. No. 6,737,056; WO 2004/056312; Shields, R. L., et al., J. Biol. Chem. 9 (2001) 6591-6604).

In certain embodiments, the antibody comprises an Fc-region with one or more amino acid substitutions which improve ADCC, e.g., substitutions at positions 298, 333, and/or 334 of the Fc-region (EU numbering of residues).

In some embodiments, alterations are made in the Fc-region of the antibody that result in altered (i.e., either improved or diminished) C1q binding and/or Complement Dependent Cytotoxicity (CDC), e.g., as reported in U.S. Pat. No. 6,194,551, WO 99/51642, and Idusogie, E. E. et al., J. Immunol. 164 (2000) 4178-4184.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593 and Kim, J. K. et al., Eur. J. Immunol. 24 (1994) 2429-2434), are reported in US 2005/0014934. Those antibodies comprise an Fc-region with one or more substitutions therein which improve binding of the Fc-region to FcRn. Such Fc variants include those with substitutions at one or more of Fc-region residues: 238, 256, 265, 272, 286, 303, 305, 307, 311, 312, 317, 340, 356, 360, 362, 376, 378, 380, 382, 413, 424 or 434, e.g., substitution of Fc-region residue 434 (U.S. Pat. No. 7,371,826).

See also Duncan, A. R. and Winter, G., Nature 332 (1988) 738-740; U.S. Pat. No. 5,648,260; U.S. Pat. No. 5,624,821; and WO 94/29351 concerning other examples of Fe-region variants.

### B. Recombinant Methods and Compositions

Fusion proteins and antibodies may be produced using recombinant methods and compositions, e.g., as reported in U.S. Pat. No. 4,816,567. In one embodiment, one or more isolated nucleic acids encoding a fusion protein as reported herein are provided. Such nucleic acid may encode an amino acid sequence comprising the VL and/or an amino acid sequence comprising the VH of the antibody (e.g., the light and/or heavy chains of the antibody). In one embodiment, one or more vectors (e.g., expression vectors) comprising such nucleic acid are provided. In one embodiment, a host cell comprising such nucleic acid is provided. In one such embodiment, a host cell comprises (e.g. has been transformed or transfected with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or a Baby Hamster Kidney (BHK) cell or a Human Embryonic Kidney (HEK) cell, or lymphoid cell (e.g. Y0, NS0, Sp2/0 cell). In one embodiment, a method of making a fusion protein as reported herein is provided, wherein the method comprises culturing a host cell comprising a nucleic acid encoding the fusion protein, as provided above, under conditions suitable for expression of the fusion protein, and optionally recovering the fusion protein from the host cell (or host cell culture medium).

For recombinant production of a fusion protein as reported herein, nucleic acid encoding the fusion protein, e.g., as described above, is isolated and inserted into one or more vectors for further cloning and/or expression in a host cell. Such nucleic acid may be readily isolated and sequenced using conventional procedures.

Suitable host cells for cloning or expression of fusion protein-encoding vectors include prokaryotic or eukaryotic cells as reported herein. For example, the fusion protein may be produced in bacteria, in particular when glycosylation and Fc effector function are not needed. For expression of fragments and polypeptides in bacteria, see, e.g., U.S. Pat. No. 5,648,237, U.S. Pat. No. 5,789,199, and U.S. Pat. No. 5,840,523, also see Charlton, Methods in Molecular Biology, Vol. 248, Lo, B. K. C. (ed.), Humana Press, Totowa, N.J., (2003), pp. 245-254, reporting expression of antibody fragments in E. coli. After expression, the fusion protein may be isolated from the bacterial cell paste in a soluble fraction and can be further purified.

In addition to prokaryotes, eukaryotic microbes such as filamentous fungi or yeast are suitable cloning or expression hosts for fusion protein-encoding vectors, including fungi and yeast strains whose glycosylation pathways have been “humanized”, resulting in the production of a fusion protein with a partially or fully human glycosylation pattern (see Gerngross, T. U., Nat. Biotech. 22 (2004) 1409-1414; L1, H. et al., Nat. Biotech. 24 (2006) 210-215).

Suitable host cells for the expression of glycosylated fusion proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see, e.g., U.S. Pat. No. 5,959,177, U.S. Pat. No. 6,040,498, U.S. Pat. No. 6,420,548, U.S. Pat. No. 7,125,978, and U.S. Pat. No. 6,417,429 (reporting PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7), human embryonic kidney line (293 or 293 cells as reported, e.g., in Graham, F. L. et al., J. Gen Virol. 36 (1977) 59-74), baby hamster kidney cells (BHK), mouse sertoli cells (TM4 cells as reported, e.g., in Mather, J. P., Biol. Reprod. 23 (1980) 243-252), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TR1 cells, as reported, e.g., in Mather, J. P. et al., Annals N.Y. Acad. Sci. 383 (1982) 44-68, MRC 5 cells, and FS4 cells. Other useful mammalian host cell lines include Chinese hamster ovary (CHO) cells, including DHFR⁻ CHO cells (Urlaub, G. et al., Proc. Natl. Acad. Sci. USA 77 (1980) 4216-4220), and myeloma cell lines such as Y0, NS0 and Sp2/0. For a review of certain mammalian host cell lines suitable for fusion protein production, see, e.g., Yazaki, P. J. and Wu, A. M., Methods in Molecular Biology, Vol. 248 (B. K. C. Lo, ed., Humana Press, Totowa, N.J.), pp. 255-268 (2003).

### C. Pharmaceutical Formulations

Pharmaceutical formulations of a fusion protein as reported herein are prepared by mixing a fusion protein having the desired degree of purity with one or more optional pharmaceutically acceptable carriers (Remington's Pharmaceutical Sciences, 16th ed., Osol, A. (ed.) (1980)), in the form of lyophilized formulations or aqueous solutions. Pharmaceutically acceptable carriers are generally non-toxic to recipients at the dosages and concentrations employed, and include, but are not limited to: buffers such as phosphate, citrate, and other organic acids, antioxidants including ascorbic acid and methionine, preservatives (such as octadecyl dimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight (less than about 10 residues) polypeptides, proteins, such as serum albumin, gelatin, or immunoglobulins, hydrophilic polymers such as poly vinylpyrrolidone, amino acids such as glycine, glutamine, asparagine, histidine, arginine, or lysine, monosaccharides, disaccharides, and other carbohydrates including glucose, mannose, or dextrins, chelating agents such as EDTA, sugars such as sucrose, mannitol, trehalose or sorbitol, salt-forming counter-ions such as sodium, metal complexes (e.g. Zn-protein complexes), and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are reported in US 2005/0260186 and US 2006/0104968.

In one aspect, a sHASEGP is combined with one or more additional glycosaminoglycanases such as chondroitinases.

Exemplary lyophilized antibody formulations are reported in U.S. Pat. No. 6,267,958. Aqueous antibody formulations include those reported in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethyl cellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th ed., Osol, A. (ed.) (1980).

Sustained-release preparations may be prepared. Suitable examples of sustained-release preparations include semi permeable matrices of solid hydrophobic polymers containing the fusion protein, which matrices are in the form of shaped articles, e.g. films, or microcapsules.

The formulations to be used for in vivo administration are generally sterile. Sterility may be readily accomplished, e.g., by filtration through sterile filtration membranes.

### D. Therapeutic Methods and Compositions

Any of the fusion proteins provided herein may be used in therapeutic methods.

In one aspect, the invention provides for the use of a fusion protein in the manufacture or preparation of a medicament.

In one aspect, the invention provides a method for treating hepatitis-B-virus infection.

In one aspect, the invention provides pharmaceutical formulations comprising any of the fusion proteins provided herein, e.g., for use in any of the above therapeutic methods. In one embodiment, a pharmaceutical formulation comprises any of the fusion proteins provided herein and a pharmaceutically acceptable carrier. In another embodiment, a pharmaceutical formulation comprises any of the fusion proteins provided herein and at least one additional therapeutic agent, e.g., as described below.

Fusion proteins of the invention can be used either alone or in combination with other agents in a therapy. For instance, a fusion protein of the invention may be co-administered with at least one additional therapeutic agent.

Such combination therapies noted above encompass combined administration (where two or more therapeutic agents are included in the same or separate formulations), and separate administration, in which case, administration of the fusion protein of the invention can occur prior to, simultaneously, and/or following, administration of the additional therapeutic agent and/or adjuvant.

A fusion protein of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion proteins of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion protein need not be, but is optionally formulated with one or more agents currently used to prevent or treat the disorder in question. The effective amount of such other agents depends on the amount of fusion protein present in the formulation, the type of disorder or treatment, and other factors discussed above. These are generally used in the same dosages and with administration routes as described herein, or about from 1% to 99% of the dosages described herein, or in any dosage and by any route that is empirically/clinically determined to be appropriate.

For the prevention or treatment of disease, the appropriate dosage of a fusion protein of the invention (when used alone or in combination with one or more other additional therapeutic agents) will depend on the type of disease to be treated, the severity and course of the disease, whether the fusion protein is administered for preventive or therapeutic purposes, previous therapy, the patient's clinical history and response to the fusion protein, and the discretion of the attending physician. The fusion protein is suitably administered to the patient at one time or over a series of treatments. Depending on the type and severity of the disease, about 1 μg/kg to 15 mg/kg (e.g. 0.1 mg/kg-10 mg/kg) of fusion protein can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered.

### E. Articles of Manufacture

In one aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a fusion protein as reported herein. The label or package insert indicates that the composition is used for treating the condition of choice. Moreover, the article of manufacture may comprise (a) a first container with a composition contained therein, wherein the composition comprises a fusion protein as reported herein, and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as water for injection (WFI), bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. Description of the Sequences

SEQ ID NO: 01 hepatitis-B-virus envelope protein amino acid sequence (hepatitis-B-virus genotype C subtype adr (isolate Japan/A4/1994) (HBV-C))
SEQ ID NO: 02 hepatitis-B-virus core protein amino acid sequence (hepatitis-B-virus genotype C subtype adr (isolate Japan/A4/1994) (HBV-C))
SEQ ID NO: 03 mature human interferon α-2a amino acid sequence
SEQ ID NO: 04 CDR-H1 amino acid sequence of c18/A2 mAb
SEQ ID NO: 05 CDR-H2 amino acid sequence of c18/A2 mAb
SEQ ID NO: 06 CDR-H3 amino acid sequence of c18/A2 mAb
SEQ ID NO: 07 murine heavy chain variable domain amino acid sequence
SEQ ID NO: 08 CDR-L1 amino acid sequence of c18/A2 mAb
SEQ ID NO: 09 CDR-L2 amino acid sequence of c18/A2 mAb
SEQ ID NO: 10 CDR-L3 amino acid sequence of c18/A2 mAb
SEQ ID NO: 11 murine light chain variable domain amino acid sequence
SEQ ID NO: 12 chimeric murine-human heavy chain amino acid sequence of c18/A2 mAb
SEQ ID NO: 13 chimeric murine-human amino acid sequence of the C-terminal c18/A2 antibody heavy chain interferon-α2a antibody fusion
SEQ ID NO: 14 chimeric murine-human light chain amino acid sequence of c18/A2 mAb
SEQ ID NO: 15 chimeric murine-human amino acid sequence of the C-terminal c18/A2 antibody light chain interferon-α2a antibody fusion protein
SEQ ID NO: 16 human Ig kappa light chain constant domain amino acid sequence
SEQ ID NO: 17 human Ig lambda light chain constant domain amino acid sequence
SEQ ID NO: 18 human IgG1 constant region (caucasian allotype) amino acid sequence
SEQ ID NO: 19 human IgG1 constant region (afroamerican allotype) amino acid sequence
SEQ ID NO: 20 human IgG1 constant region variant amino acid sequence
SEQ ID NO: 21 human IgG4 constant region amino acid sequence
SEQ ID NO: 22 human IgG4 constant region variant amino acid sequence
SEQ ID NO: 23 linker 1 amino acid sequence
SEQ ID NO: 24 linker 2 amino acid sequence
SEQ ID NO: 25 linker 3 amino acid sequence
SEQ ID NO: 26 linker 4 amino acid sequence
SEQ ID NO: 27 linker 5 amino acid sequence
SEQ ID NO: 28 linker 6 amino acid sequence
SEQ ID NO: 29 HBV-envelope derived peptidic fragment
SEQ ID NO: 30 HBV-core derived peptidic fragment
SEQ ID NO: 31 HBV-envelope derived peptidic fragment
SEQ ID NO: 32 CDR-H1 amino acid sequence of e183/A2 mAb
SEQ ID NO: 33 CDR-H2 amino acid sequence of e183/A2 mAb
SEQ ID NO: 34 CDR-H3 amino acid sequence of e183/A2 mAb
SEQ ID NO: 35 murine heavy chain variable domain amino acid sequence of antibody against HBV envelope peptidic fragment of amino acid residues 182 to 190 of SEQ ID NO: 01
SEQ ID NO: 36 CDR-L1 amino acid sequence of e183/A2 mAb
SEQ ID NO: 37 CDR-L2 amino acid sequence of e183/A2 mAb
SEQ ID NO: 38 CDR-L3 amino acid sequence of e183/A2 mAb
SEQ ID NO: 39 murine light chain variable domain amino acid sequence of antibody against HBV envelope peptidic fragment of amino acid residues 182 to 190 of SEQ ID NO: 01

### IV. Examples

The following are examples of methods and compositions of the invention. It is understood that various other embodiments may be practiced, given the general description provided above.

### Materials & Methods

General information regarding the nucleotide sequences of human immunoglobulins light and heavy chains is given in: Rabat, E. A., et al., (1991) Sequences of Proteins of Immunological Interest, 5th ed., vols. 1-3, Public Health Service, NIH Publication No 91-3242.

Amino acids of antibody chains are numbered according to EU numbering (Edelman, G. M., et al., PNAS 63 (1969) 78-85; Kabat, E. A., et al., (1991) Sequences of Proteins of Immunological Interest, 5th ed., vols. 1-3, Public Health Service, NIH Publication No 91-3242).

### Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y. (1989). The molecular biological reagents were used according to the manufacturer's instructions.

### DNA Sequence Determination

DNA sequences were determined by double strand sequencing performed at SequiServe GmbH (Vaterstetten, Germany). DNA and protein sequence analysis and sequence data management The GCG's (Genetics Computer Group, Madison, Wis.) software package variant 10.2 and Infomax's Vector NTI Advance suite variant 8.0 was used for sequence creation, mapping, analysis, annotation and illustration.

### Gene Synthesis

Desired gene segments encoding the heavy and light chain variable domain of the mouse c18/A2 mAb and e183/A2 mAb were prepared by Geneart GmbH (Regensburg, Germany). The gene segments are flanked by singular restriction endonuclease cleavage sites to facilitate expression construct cloning as described below. The DNA sequence of the subcloned gene fragments were confirmed by DNA sequencing.

### Example 1

### Generation of the Expression Plasmids for the Chimeric Murine-Human c18/a2 TCR-like antibody interferon-α2a fusion protein

The chimeric murine-human c18/A2 TCR-like antibody heavy chain interferon-α2a fusion gene was assembled by fusing a chemically synthesized DNA fragment coding for mature human IFN-α2a and a glycine-serine linker consisting of two Gly4Ser repeats (heavy chain . . . LSPG—GGGSGGGGS—IFNa2a) to the 3′ end of the c18/A2 TCR-like antibody heavy chain gene coding for a slightly truncated human gamma-1 heavy chain constant region (removal of the last natural amino acid Lys).

Generation of the Expression Plasmids for the Chimeric Murine-Human c18/a2 TCR-Like Parental Antibody

The gene segments encoding the mouse c18/A2 TCR-like mAb kappa light (VK) and heavy chain variable regions (VH) were joined to the gene segments encoding the human kappa light chain constant region (CK) or the human gamma-1 heavy chain constant region (CH1-Hinge-CH2-CH3), respectively. Both antibody chain genes were expressed from two separate expression plasmids including the genomic exon-intron structure of the antibody genes.

The expression of antibody chains is controlled by a shortened intron A-deleted immediate early enhancer and promoter from the human cytomegalovirus (HCMV) including a human heavy chain immunoglobulin 5′-untranslated region (UTR), a murine immunoglobulin heavy chain signal sequence, and the strong polyadenylation signal from bovine growth hormone. The expression plasmids also contain an origin of replication and a β-lactamase gene from the vector pUC18 for plasmid amplification in Escherichia coli and an optional neomycin resistance gene for the generation/selection of stably transfected mammalian cell lines.

### a) Plasmid 9924

Plasmid 9924 is the expression plasmid for the transient expression of chimeric murine-human c18/A2 TCR-like antibody γ1-heavy chain IFN-α2a fusion protein (genomically organized expression cassette; exon-intron organization) in HEK293 cells.

Besides the c18/A2 TCR-like antibody yl-heavy chain IFN-α2a expression cassette this vector contains:
an origin of replication from the vector pUC 18 which allows replication of this plasmid in E. coli, and
a beta-lactamase gene which confers ampicillin resistance in E. coli.

The transcription unit for the c18/A2 TCR-like antibody γ1-heavy chain IFN-α2a fusion gene coding for the mature c18/A2 TCR-like antibody γ1-heavy chain IFN-α2a fusion protein as given in SEQ ID NO: 13—comprises the following elements:
the immediate early enhancer and promoter from the human cytomegalovirus (CMV),
a human heavy chain immunoglobulin 5′-untranslated region (UTR),
a murine immunoglobulin heavy chain signal sequence including a signal sequence intron (signal sequence 1, intron, signal sequence 2 [L1-intron-L2]),
the variable heavy chain encoding segment (SEQ ID NO: 07) arranged with a unique BsmI restriction site at the 5′-end (L2 signal sequence) and a splice donor site and a unique XhoI restriction site at the 3′-end,
a truncated mouse/human heavy chain hybrid intron 2 including the mouse heavy chain enhancer element (part JH3, JH4) (see e.g. Neuberger, M. S., EMBO J. 2 (1983) 1373-1378),
the human γ1-heavy gene constant region in genomic organization from which the last codon encoding the C-terminal Lys has been deleted,
a glycine-serine linker (SEQ ID NO: 23)
the mature human IFNa2a gene (SEQ ID NO: 03) and
the bovine growth hormone polyadenylation (BGH pA) signal sequence.

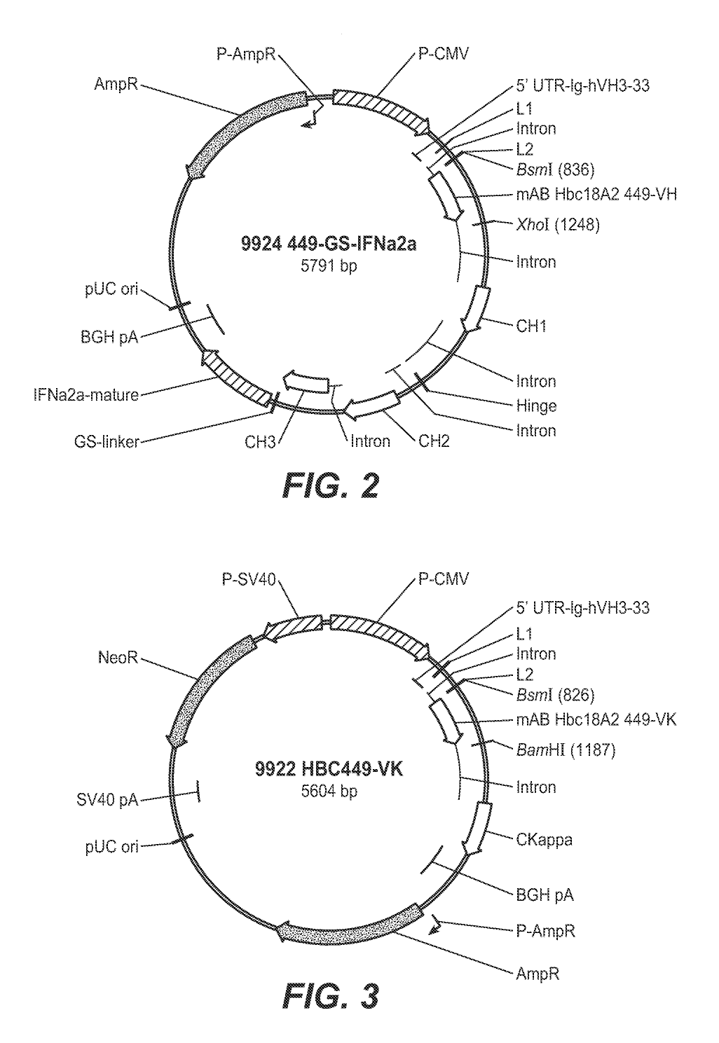

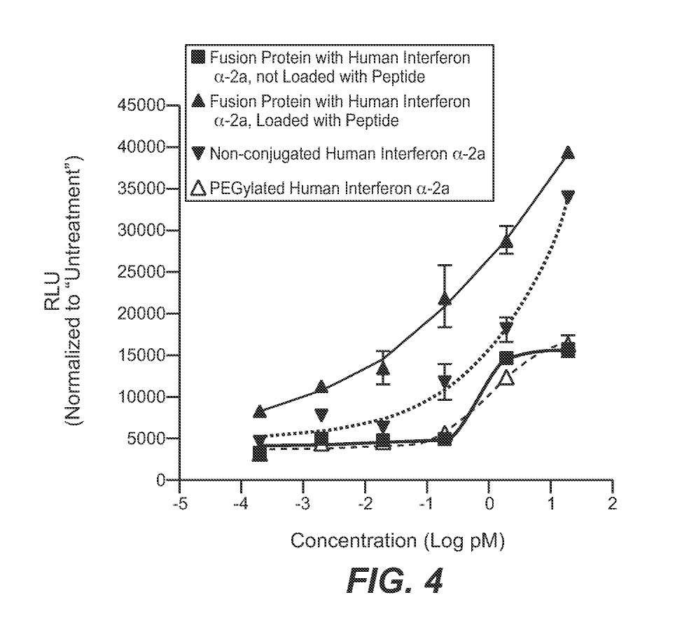

The plasmid map of the heavy chain expression plasmid 9924 is shown in FIG. 2.

### b) Plasmid 9922

Plasmid 9922 is the expression plasmid for the transient expression of the chimeric murine-human c18/A2 TCR-like antibody light chain (genomically organized expression cassette; exon-intron organization) in HEK293 cells.

Beside c18/A2 TCR-like antibody ic-light chain expression cassette this vector contains:
an SV40 promoter
a neomycin resistance gene as a selectable marker,
an origin of replication from the vector pUC 18 which allows replication of this plasmid in E. coli, and
a β-lactamase gene which confers ampicillin resistance in E. coli.

The transcription unit for the c18/A2 TCR-like antibody x-light chain gene—coding for the mature c18/A2 TCR-like antibody κ-light chain protein as given in SEQ ID NO: 14—is composed of the following elements:
the immediate early enhancer and promoter from the human cytomegalovirus (CMV),
a human heavy chain immunoglobulin 5′-untranslated region (UTR),
a murine immunoglobulin heavy chain signal sequence including a signal sequence intron (signal sequence 1, intron, signal sequence 2 [L1-intron-L2]),
the variable light chain encoding segment (SEQ ID NO: 11) arranged with a unique BsmI restriction site at the 5′-end (L2 signal sequence) and a splice donor site and a unique BamHI restriction site at the 3′-end,
a truncated human kappa light chain intron 2
the human kappa light chain gene constant region, and
the bovine growth hormone polyadenylation (BGH pA) signal sequence.

The plasmid map of the light chain expression plasmid 9922 is shown in FIG. 3.

### Example 2

### Generation of the Expression Plasmids for the Chimeric Murine-Human e183/a2 TCR-Like Antibody IFN-α2a Fusion Protein

The chimeric murine-human e183/A2 TCR-L antibody IFN-α2a fusion genes were assembled in the same way as described for the chimeric murine-human c18/A2 TCR-like antibody IFN-α2a fusion genes resulting in the expression plasmids 9976 (antibody heavy chain-IFN-α2a fusion gene) 9977 (antibody light chain gene).

### Example 3

### Transient Expression, Purification and Analytical Characterization of Immunoglobulin-Interferon Alpha Fusion Proteins in HEK293 Cells

Immunoglobulin-interferon alpha fusion proteins were generated by transient transfection of HEK293 cells (human embryonic kidney cell line 293-derived) cultivated in F17 Medium (Invitrogen Corp.). For transfection “293-Free” Transfection Reagent (Novagen) was used. Immunoglobulin light and heavy chains were expressed from two different plasmids using an equimolar ratio of light chain to heavy chain encoding plasmid. Transfections were performed as specified in the “293-Free” manufacturer's instructions. Fusion protein-containing cell culture supernatants were harvested 7 days after transfection. Supernatants were stored at reduced temperature until purification.

General information regarding the recombinant expression of human immunoglobulins in e.g. HEK293 cells is given in: Meissner, P. et al., Biotechnol. Bioeng. 75 (2001) 197-203.

Antibody-containing culture supernatants were filtered and purified by two chromatographic steps. Antibodies were captured by affinity chromatography using Protein A Sepharose™ CL-4B (GE Healthcare) equilibrated with 0.1 M phosphate buffer, pH 7.0. Unbound proteins were washed out with equilibration buffer, and the antibodies were eluted with 0.1M citrate buffer, pH 3.5, and then immediately neutralized to pH 6.0 with 1 M Tris-base. Size exclusion chromatography on Superdex 200™ (GE Healthcare) was used as a second purification step. Size exclusion chromatography was performed in 20 mM histidine buffer, 0.14 M NaCl, pH 6.0. The eluted antibodies were concentrated with an Ultrafree-CL centrifugal filter unit equipped with a Biomax-SK membrane (Millipore, Billerica, Mass.) and stored at −80° C.

The protein concentration of antibodies and antibody fusions was determined by measuring the optical density (OD) at 280 nm, using the molar extinction coefficient calculated on the basis of the amino acid sequence. Purity and proper tetramer formation of antibodies and antibody fusions were analyzed by SDS-PAGE in the presence and absence of a reducing agent (5 mM 1,4-dithiotreitol) and staining with Coomassie brilliant blue. Aggregate content of antibodies and antibody fusions preparations was analyzed by high-performance SEC using a SK3000SW×1 analytical size-exclusion column (Tosohaas, Stuttgart, Germany). The integrity of the amino acid backbone of reduced antibodies and antibody fusions light and heavy chains were verified by Nano Electrospray QTOF mass spectrometry after removal of N-glycans by enzymatic treatment with Peptide-N-Glycosidase F (Roche Molecular Biochemicals).

### Example 4

### Determination of the Binding Affinity

Amine coupling of around 750 resonance units (RU) of a capturing system (capturing mAb specific for human IgG, Jackson Immunoresearch) was performed on a CM5 chip at pH 4.5 using an amine coupling kit supplied by the GE Healthcare. HuFc-tagged IFNAR2 (RnD Systems, Cat-Nr. 4015-AB) was captured at a concentration of 5 μg/ml. Excess binding sites were blocked by injecting a huFc mixture at a concentration of 1.25 μM (Biodesign, Cat-Nr. 50175). Different concentrations of Interferon or Interferon fusions ranging from 0.1 nM to 50 nM were passed with a flow rate of 10 μl/min through the flow cells at 298 K for 120-240 sec. to record the association phase. The dissociation phase was monitored for up to 600 sec. and triggered by switching from the sample solution to running buffer. The surface was regenerated by 1 min. washing with a 100 mM phosphoric acid solution at a flow rate of 30 μl/min. For all experiments HBS-P+buffer supplied by GE Healthcare was chosen (10 mM HEPES ((4-(2-hydroxyethyl)-1-piperazine ethanesulfonic acid)), pH 7.4, 150 mM NaCl, 0.05% (v/v) Surfactant P20).

Bulk refractive index differences were corrected for by subtracting the response obtained from a blank-coupled surface. Blank injections are also substracted (=double referencing).

The equilibrium dissociation constant (Kd), defined as ka/kd, was determined by analyzing the sensogram curves obtained with several different concentrations, using BIAevaluation 4.1 software package. The fitting of the data followed a suitable binding model.

For wildtype IFN α-2a 0.1 nM to 50 nM IFN α-2a was injected over an IFNAR2 coated sensor chip as shown in FIG. 1 a). For IFN α-2a fused C-terminally to a huFc fragment, such a protein was injected at a concentration of 0.5 to 50 nM over an IFNAR2 coated surface. Due to bivalent binding complex stability increases from 35 sec. for IFN α-2a to 23 min. for Fc-IFN α-2a fusions. Respectively, the affinity increases from 4 nM for IFN α-2a to an apparent affinity of 0.3 nM. Since for activity IFNAR1 is essential only initial binding can be addressed no interferon signaling activity by such an assay.

1. A fusion protein comprising an antibody that specifically binds to a human major histocompatibility complex presenting a peptidic fragment of a hepatitis-B-virus protein and an anti-viral cytokine.

2. The fusion protein according to claim 1, wherein the peptidic fragment of the hepatitis-B-virus protein has an amino acid sequence selected from the group consisting of the amino acid sequence of amino acid residues 182 to 190 of SEQ ID NO: 01 and the amino acid sequence of amino acid residues 18 to 27 of SEQ ID NO: 02.

3. The fusion protein according to claim 1, wherein the antibody specifically binds to hepatocytes infected with hepatitis-B-virus.

4. The fusion protein according to claim 1, wherein the anti-viral cytokine is selected from the group consisting of a type I interferon and a type II interferon.

5. The fusion protein according to claim 1, wherein the fusion protein has the same specificity as CD 8 bearing T-cells.

6. The fusion protein according to claim 1, wherein the antibody does not specifically bind to serum hepatitis-B-virus antigens.

7. The fusion protein according to claim 1, wherein the antibody is a monoclonal antibody.

8. The fusion protein according to claim 7, wherein the antibody is a human, humanized, or chimeric antibody.

9. The fusion protein according to claim 7, wherein the antibody is an antibody fragment that binds a human major histocompatibility complex presenting a peptidic fragment of a hepatitis-B-virus protein.

10. The fusion protein according to claim 1, wherein the antibody comprises heavy and light chain CDR amino acid sequences, wherein (a) CDR-H1 comprises the amino acid sequence of SEQ ID NO: 04, (b) CDR-H2 comprises the amino acid sequence of SEQ ID NO: 05, (c) CDR-H3 comprises the amino acid sequence of SEQ ID NO: 06, (d) CDR-L1 comprises the amino acid sequence of SEQ ID NO: 8, (e) CDR-L2 comprises the amino acid sequence of SEQ ID NO: 9, and (f) CDR-L3 comprises the amino acid sequence of SEQ ID NO: 10.

11. The fusion protein according to claim 1, wherein the antibody comprises heavy and light chain CDR amino acid sequences, wherein (a) CDR-H1 comprising the amino acid sequence of SEQ ID NO: 32, (b) CDR-H2 comprises the amino acid sequence of SEQ ID NO: 33, (c) CDR-H3 comprises the amino acid sequence of SEQ ID NO: 34, (d) CDR-L1 comprises the amino acid sequence of SEQ ID NO: 36, (e) CDR-L2 comprises the amino acid sequence of SEQ ID NO: 37, and (f) CDR-L3 comprises the amino acid sequence of SEQ ID NO: 38.

12. The fusion protein according to claim 1, wherein the antibody comprises a variable heavy chain and a variable light chain, wherein further the variable heavy chain amino acid sequence has at least 95% sequence identity to the amino acid sequence of SEQ ID NO: 07 or the variable light chain amino acid sequence has at least 95% sequence identity to the amino acid sequence of SEQ ID NO: 11, or to a humanized variant thereof.

13. The fusion protein according to claim 1, wherein the antibody comprises a variable heavy chain and a variable light chain, wherein further the variable heavy chain amino acid sequence has at least 95% sequence identity to the amino acid sequence of SEQ ID NO: 35 or the variable light chain amino acid sequence has at least 95% sequence identity to the amino acid sequence of SEQ ID NO: 39, or to a humanized variant thereof.

14. The fusion protein according to claim 12, wherein the variable heavy chain amino acid sequence comprises the amino acid sequence of SEQ ID NO: 07, or a humanized variant thereof.

15. The fusion protein according to claim 12, wherein the variable light chain amino acid sequence comprises the amino acid sequence of SEQ ID NO:11.

16. The fusion protein according to claim 13, wherein the variable light chain amino acid sequence comprises the amino acid sequence of SEQ ID NO: 39, or a humanized variant thereof.

17. The fusion protein according to claim 13, wherein the variable heavy chain amino acid sequence comprises the amino acid sequence of SEQ ID NO:35.

18. The fusion protein according to claim 12, wherein the antibody heavy chain comprises the amino acid sequence of SEQ ID NO:12.

19. The fusion protein according to claim 12, wherein the antibody light chain comprises the amino acid sequence of SEQ ID NO: 14.

20. The fusion protein according to claim 13, wherein the antibody heavy chain comprises the amino acid sequence of SEQ ID NO:35.

21. The fusion protein according to claim 13, wherein the antibody light chain comprises the amino acid sequence of SEQ ID NO:39.

22. The fusion protein according to claim 12 comprising a human antibody constant region.

23. The fusion protein according to claim 13 comprising a human antibody constant region

24. An isolated nucleic acid encoding the fusion protein of claim 1.

25. A host cell comprising the nucleic acid of claim 24.

26. A process for producing a fusion protein, comprising the steps of:
(a) providing the host cell according to claim 25,
(b) cultivating the cell of (a),
(c) recovering the fusion protein from the cell or the cultivation medium and thereby producing the fusion protein.

27. A pharmaceutical formulation comprising the fusion protein of any one of claims 1 to 23 and a pharmaceutically acceptable carrier.

28. A method of treating an individual having a hepatitis-B-virus infection comprising administering to the individual an effective amount of the fusion protein of claim 1.

29. A method of delivering an anti-viral cytokine to hepatitis-B-virus infected hepatocytes in an individual comprising administering to the individual an effective amount of the fusion protein of claim 1.


SEQUENCE LISTING (39 sequences)

SEQ ID NO: 1 - protein, 388 aa, Hepatitis B virus
GTNLSVPNPLGFFPDHQLDPAFGANSNNPDWDFNPNKDQWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTIVSPISSIFSRTGDPAPNMESTTSGFLGPLLVLQAGFFLLTRILTIPQSLDSWWTSLNFLGEAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFAGLSPTVWLSVIWMMWYWGPSLYNILSPFLPLLPIFFCLWVYI

SEQ ID NO: 2 - protein, 183 aa, Hepatitis B virus
MDIDPYKEFGATVELLSFLPSDFFPSVRDLLDTAAALYREALESPEHCSPHHTALRQAILCWVELMTLATWVGNNLEDPASRDLVVNYVNTNMGLKIRQLLWFHISCLTFGRETVLEYLVSFGVWIGTPPAYRPPNAPILSTLPETTVVRRRGRSPRRRTPSPRRRRSQSPRRRRSQSRESQC

SEQ ID NO: 3 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 4 - protein, 5 aa, Mus musculus
SYDIN

SEQ ID NO: 5 - protein, 17 aa, Mus musculus
WIYPGDGSTKYNEKFKG

SEQ ID NO: 6 - protein, 11 aa, Mus musculus
RDYGYSYAMDY

SEQ ID NO: 7 - protein, 120 aa, Mus musculus
QVQLQQSGPELVKPGASVKISCKASGHTFTSYDINWVKQRPGQGLEWIGWIYPGDGSTKYNEKFKGKATLTADKSSSTAYMQLSSLTSENSAVYFCARRDYGYSYAMDYWGQGTSVTVSS

SEQ ID NO: 8 - protein, 11 aa, Mus musculus
RASGNIHNYLA

SEQ ID NO: 9 - protein, 7 aa, Mus musculus
NAKTLAD

SEQ ID NO: 10 - protein, 9 aa, Mus musculus
QHFWSTPFT

SEQ ID NO: 11 - protein, 107 aa, Mus musculus
DIQMTQSPASLSTSVGETVTITCRASGNIHNYLAWYQQKQGKSPQLLVYNAKTLADGVPSRFSGSGSGTQFSLKINSLQPEDFGSYYCQHFWSTPFTFGSGTKLEIK

SEQ ID NO: 12 - protein, 448 aa, Artificial Sequence
  chimeric mouse-human heavy chain
QVQLQQSGPELVKPGASVKISCKASGHTFTSYDINWVKQRPGQGLEWIGWIYPGDGSTKYNEKFKGKATLTADKSSSTAYMQLSSLTSENSAVYFCARRDYGYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 13 - protein, 624 aa, Artificial Sequence
  chimeric mouse-human heavy chain IFNa2a fusion
QVQLQQSGPELVKPGASVKISCKASGHTFTSYDINWVKQRPGQGLEWIGWIYPGDGSTKYNEKFKGKATLTADKSSSTAYMQLSSLTSENSAVYFCARRDYGYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 14 - protein, 214 aa, Artificial Sequence
  chimeric mouse-human light chain
DIQMTQSPASLSTSVGETVTITCRASGNIHNYLAWYQQKQGKSPQLLVYNAKTLADGVPSRFSGSGSGTQFSLKINSLQPEDFGSYYCQHFWSTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 390 aa, Artificial Sequence [sequence omitted]

SEQ ID NO: 16 - protein, 107 aa, Homo sapiens [sequence omitted]

SEQ ID NO: 17 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 330 aa, Homo sapiens [sequence omitted]

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 11 aa, Artificial Sequence
  linker 1
GGGGSGGGGSG

SEQ ID NO: 24 - protein, 16 aa, Artificial Sequence
  linker 2
GGGGSGGGGSGGGGSG

SEQ ID NO: 25 - protein, 21 aa, Artificial Sequence
  linker 3
GGGGSGGGGSGGGGSGGGGSG

SEQ ID NO: 26 - protein, 9 aa, Artificial Sequence
  linker 4
GGGSGGGSG

SEQ ID NO: 27 - protein, 13 aa, Artificial Sequence
  linker 5
GGGSGGGSGGGSG

SEQ ID NO: 28 - protein, 17 aa, Artificial Sequence
  linker 6
GGGSGGGSGGGSGGGSG

SEQ ID NO: 29 - protein, 9 aa, Hepatitis B virus
FLLTRILTI

SEQ ID NO: 30 - protein, 10 aa, Hepatitis B virus
FLPSDFFPSV

SEQ ID NO: 31 - protein, 9 aa, Hepatitis B virus
FLLTRILTI

SEQ ID NO: 32 - protein, 5 aa, Mus musculus
SYWMH

SEQ ID NO: 33 - protein, 17 aa, Mus musculus
EIDPSDSYTNYNQKFKG

SEQ ID NO: 34 - protein, 9 aa, Mus musculus
SGYDVGVDY

SEQ ID NO: 35 - protein, 118 aa, Mus musculus
QVQLQQPGAEFVKPGASVRLSCKASGYTFSSYWMHWVKQRPGQGLEWIGEIDPSDSYTNYNQKFKGKATLTVDKSSSTAYMQFSSLTSEDSAVYYCATSGYDVGVDYWGQGTTLTVSS

SEQ ID NO: 36 - protein, 11 aa, Mus musculus
RASENIYSYLA

SEQ ID NO: 37 - protein, 7 aa, Mus musculus
IAKTLAE

SEQ ID NO: 38 - protein, 9 aa, Mus musculus
QHHYGTPYT

SEQ ID NO: 39 - protein, 107 aa, Mus musculus
DIQMTQSPASLSASVGETVTITCRASENIYSYLAWYHQKQGKSPQLLVYIAKTLAEGVPSRFSGSGSGTQFSLKIDSLQPEDVGSYYCQHHYGTPYTFGGGTKLEIK